SEQUENCE LISTING (7 sequences)

SEQ ID NO: 1 - DNA, 972 bp, Homo sapiens
  source 1..972: mol_type = unassigned DNA; note = mRNA Human CD47 coding sequence (CDS) of isoform 1
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 2 - DNA, 918 bp, Homo sapiens
  source 1..918: mol_type = unassigned DNA; note = mRNA Human CD47 CDS of isoform 2
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 3 - DNA, 939 bp, Homo sapiens
  source 1..939: mol_type = unassigned DNA; note = mRNA Human CD47 CDS of isoform 3
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataa

SEQ ID NO: 4 - DNA, 1515 bp, Homo sapiens
  source 1..1515: mol_type = unassigned DNA; note = mRNA Human SIRPA CDS of isoform 1
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 5 - DNA, 1527 bp, Homo sapiens
  source 1..1527: mol_type = unassigned DNA; note = mRNA Human SIRPA CDS of isoform 2
atggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggtacagtccttggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtga

SEQ ID NO: 6 - DNA, 2374 bp, synthetic construct
  source 1..2374: mol_type = other DNA
aactacttaggtgaaagtgttgataaatggctgaagaagatggaagacaactacagtgtctccacatttttgcacattttaggtgaaagatgagtcatcacatagactttcttaatccctcttctataaagcaacagccagtggtttagcctatgggagagttgataggatatatgttacttattagccatatctactgttggtttcaacttgcgtcttagttttattgtgggttgtttgtggtttctcaaaggaaggaacacttgtctgtgcatttggaagtttttccttggtttcattgaaatgtgggagtgggaaaatgccattcagttgtgtataatcctttaaggtacttgcatacaaaatgtctgttgttatctgtattttgctgtgggaaagtagccatacagaatgttttctatccttagcattttaagactttccattctagtggaaagatgtgatgtgattcatgtgatttactttttatattaaagtacagtagagcctgccaccttttttggaaatgaaacatagggatagagataaacttcatatcagtttagcaagttctattttgaggaagtgtattgcagtttcgactgaatataatatttattttcagtgtaaatttttctcatggtaaggacaatatttttctacatatatcagtaggcattaatggttacttcaaagcttccaaagccagatactacactatatgttctaacatggttgctgaattttttgcccaagatgcgtgagaatttgtattttgttttactgggaaagcataaagaaaaattagtgttgtttatcagctgcgcacattaatattaaaataatttgtttcttcttttcaccctgcccatcttctcttctctgtagccaccttacttgcttaagtgaacaatcttatcttaaggatttgtagcatacccagaatcttatagtatagaattatgtaatgaaatgaaacaactttatttctctctttcaggatcagctcagctactatttaataaaacaaaatctgtagaattcacgttttgtaatgacactgtcgtcattccatgctttgttactaatatggaagcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaacacgcgaaggtgaaacgatcatcgagctaaaatatcgtgttggtaagatgtctataaagtttcccttttatttctctagcagcaacatgatcttgttagaaagcagggcacattggggttaggaactctgaatcaaaaagcaggctcctggaggtccctgttggctccgagggttaaggatgcagtgttgccactgctgtggctctggttacagctatggcgtgggttcagtccctggcctgggaacttctgcatgatgtaggcccagcaaaaaaaataaaagcaggctactaataataaattttggtgcaatgtttagcagctttagggtcatgaggttttatcttatttgctcttctacttttcacaataaatctcctttgttgcctattaatttgtacattgtgtgattctcagacttttagtttgaggactgttctattgcagtgagcagttctggggtcgtttactctgcatttctgtttgctaccatcgaaagtactttcttcctgtgattaattttcaaatcttttgcaagttatttagccttttttttttttttaaccttacactttgatcaaggattgacaaatcccagccatgcttcttctagaagaatagattaggaggagaatccaaagattaaaaattaggttatcatggctgattcttgtgcttagccttgctttggtatttttttttttcccttggccaagatgtgagagagctagggatccaatttaatttatttaaattcacccttggttataatactgtgattctgccctttctttataacttgagttagtgagttgcataaagtagtaaagctagccaaagtaaacaggctcctttcttttctattccttgtcagcttatttcttcagccacccagaaatacatcccagcttcctattttgaaagctctgtttagaatcactagacatgttaccgatctcaggattaggccattctttgggaacctgtgctttaaatgggatcagatcagttcaattagagctactatttgcctctattctctcatttattgttcctaaatgt

SEQ ID NO: 7 - DNA, 476 bp, synthetic construct
  source 1..476: mol_type = other DNA
tacccagaatcttatagtatagaattatgtaatgaaatgaaacaactttatttctctctttcaggttcagctcagttgatatttaatataaccaaatctgtagaattcaccgtttgcaatacaactgttaccatcccatgttttgttaataacatggaggcaaagaacatttctgaattgtatgtcaagtggaaatttaaaggaaaagacattttcatctttgatggcgctcagcacatatccaagcccagtgaagccttccctagttcaaaaatctcaccatcagaattactacatggcattgcctctttgaagatggataagcgtgatgccgtcataggaaactacacttgtgaagtaacagaattaagcagagaaggcgaaaccatcatagaactaaaacgtcgttttggtaagatgtctataaagtttcccttttatttctctagcagcaacatgatcttgttagaaagcag

(19) United States
(12) Patent Application Publication
(10) Pub. No.: US 2025/0098652 A1
(43) Pub. Date: Mar. 27, 2025

(54) Genetic Modifications for Xenotransplantation

(71) Applicant: The Trustees of Columbia University in the City of New York, New York, NY (US)

(72) Inventors: Megan Sykes, New York, NY (US); Robert J. Hawley, New York, NY (US)

(21) Appl. No.: 18/833,151

(22) PCT Filed: Jan. 27, 2023
(86) PCT No.: PCT/US2023/061434

§ 371 Date: Jul. 25, 2024

Related U.S. Application Data
(60) Provisional application No. 63/304,220, filed on Jan. 28, 2022.

(51) Int. Cl. A01K 67/0278; C07K 14/47; C07K 14/705; C12N 15/85

(52) U.S. Cl. CPC A01K 67/0278; C07K 14/4703; C07K 14/70596; C12N 15/8509; A01K 2207/15; A01K 2217/072; A01K 2217/15; A01K 2227/108; A01K 2267/025

(57) ABSTRACT

Provided herein are recombinant miniature swine without expression of endogenous porcine CD47 and SIRPA, but with the expression of human or humanized CD47 and human or humanized SIRPA under the same regulatory elements as the endogenous porcine CD47 and SIRPA. Also provided are cells, tissues, and organs derived from such recombinant miniature swine. Furthermore, provided herein are methods of transplanting a graft from a first donor of such recombinant miniature swine with or without bone marrow from a second donor of such recombinant miniature swine.

### 1. CROSS-REFERENCE TO RELATED APPLICATIONS

This application claims the benefit of U.S. Provisional Application No. 63/304,220, filed Jan. 28, 2022, the full disclosure of which is hereby incorporated by reference herein in its entirety.

### 2. REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14648-006-228_SequenceListing.xml,” was created on Jan. 24, 2023, and is 16,653 bytes in size.

### 3. FIELD

Provided herein are recombinant miniature swine that do not express endogenous porcine CD47 or SIRPA, and express human or humanized CD47 and human or humanized SIRPA. In certain embodiments, expression of human or humanized CD47 and human or humanized SIRPA in the recombinant miniature swine provided herein is under the same regulatory elements as the endogenous porcine CD47 and SIRPA. Also provided are cells, tissues, and organs derived from such recombinant miniature swine. Furthermore, provided herein are methods of transplanting a graft from a first donor of such recombinant miniature swine with or without bone marrow from a second donor of such recombinant miniature swine.

### 4. BACKGROUND

The severe shortage of allogeneic donors currently limits the number of organ transplants performed. This supply-demand disparity may be corrected by the use of organs from other species (xenografts). In view of the ethical issues and impracticalities associated with the use of non-human primates, pigs are considered the most suitable donor species for humans. In addition to organ size and physiologic similarities to humans, the ability to rapidly breed and inbreed pigs makes them particularly amenable to genetic modifications that could improve their ability to function as graft donors to humans. See, e.g., Sachs (1994), Path. Biol. 42:217-219 and Piedrahita et al. (2004), Am. J. Transplant, 4 Suppl. 6:43-50.

Although transplantation coupled with non-specific immunosuppressive therapy is associated with high early graft acceptance rates, a major limitation to the success of clinical organ transplantation has been late graft loss, due largely to chronic rejection of the transplant. Immune tolerance is therefore a major goal in transplantation and will be even more important for successful clinical xenotransplantation, as the level of life-long immunosuppression required to prevent xenograft rejection can be too toxic to be acceptable. In addition, no markers have been identified to reliably indicate whether immunological tolerance has been achieved in patients, resulting in an absence of laboratory parameters upon which to base immunosuppression withdrawal.

Therefore, goals in xenotransplantation include achievement of immune tolerance. This could be achieved by xenogeneic thymic transplantation or by optimizing the durability of mixed chimeric cells originated from the donor animal after they are transplanted into a xenogeneic recipient, as well as maintaining the health and viability of the donor animal.

Mixed chimerism can induce tolerance to the donor at the level of T cells, B cells and natural killer (NK) cells in the recipient. See, e.g., Griesemer et al. (2014), Immunol. Rev. 258: 241-258; Sachs et al. (2014), Cold Spring Harb. Perspect. Med. 4:a015529.

CD47, also known as integrin-associated protein (IAP), is a ubiquitously expressed 50-kDa cell surface glycoprotein and serves as a ligand for signal regulatory protein SIRPα, (also known as CD172a, and SHPS-1, whose gene is SIRPA). See, e.g., Brown (2002), Curr. Opin. Cell. Biol., 14:603-7; and Brown and Frazier (2001), Trends Cell Biol., 111130-5. CD47 and SIRPα, constitute a cell-cell communication system that plays important roles in a variety of cellular processes including cell migration, adhesion of B cells, and T cell activation. See, e.g., Liu et al. (2002), J. Biol. Chem. 277: 10028; Motegi et al. (2003), EMBO 122:2634; Yoshida et al. (2002), J. Immunol. 168:3213; and Latour et al. (2001), J. Immunol. 167:2547. In addition, the CD47-SIRPα system is implicated in negative regulation of phagocytosis by macrophages. CD47 on the surface of some cell types (i.e., erythrocytes, platelets or leukocytes) inhibited phagocytosis by macrophages. The role of CD47-SIRPα interaction in the inhibition of phagocytosis has been illustrated by the observation that primary, wild-type mouse macrophages rapidly phagocytose unopsonized red blood cells (RBCs) obtained from CD47-deficient mice but not those from wild-type mice. See, e.g., Oldenborg et al. (2000), Science 288:2051. It has also been reported that through its receptors, SIRPα, CD47 inhibits both Fc7 and complement receptor mediated phagocytosis. See, e.g., Oldenborg et al. (2001), J. Exp. Med. 193:855. CD47KO cells are vigorously rejected by macrophages after infusion into syngeneic wild-type (WT) mice, demonstrating that CD47 provides a “don't eat me” signal to macrophages. See, e.g., Oldenborg P A, et al. (2000), Science, 288:2051-4; and Wang et al. (2007), Proc Natl Acad Sci USA. 104:13744. Xenotransplantation using pigs as the transplant source has the potential to resolve the severe shortage of human organ donors, a major limiting factor in clinical transplantation. See, e.g., Yang et al. (2007), Nature Reviews Immunology. 7:519-31. The strong rejection of xenogeneic cells by macrophages (see, e.g., Abe (2002), The Journal of Immunology 168:621) is largely caused by the lack of functional interaction between donor CD47 and recipient SIRPα (see, e.g., Wang et al. (2007), Blood; 109:836-42; Ide et al. (2007), Proc Natl Acad Sci USA 104:5062-6; and Navarro-Alvarez (2014), Cell transplantation, 23:345-54), leading to the development of human CD47 transgenic pigs (see, e.g., Tena et al. (2017), Transplantation 101:316-21; and Nomura et al. (2020), Xenotransplantation 2020; 27:e12549). In addition to macrophages, a sub-population of DCs also express SIRPα (see, e.g., Wang et al. (2007), Proc Natl Acad Sci USA. 104:13744-9; and Guilliams et al. (2016), Immunity. 45:669-84). CD47-SIRPα signaling also inhibits DC activation and their ability to prime T cells, and plays an important role in induction of T cell tolerance by donor-specific transfusion (DST) or hepatocyte transplantation. See, e.g., Wang et al. (2007), Proc Natl Acad Sci USA. 104:13744-9; Wang et al. (2014), Cell transplantation 23:355-63; and Zhang et al. (2016), Sci Rep. 6:26839. Thus, there is an unmet need to generate safe and well-tolerated donor materials (e.g., organs, tissue, cells, etc.) for xenotransplantation that do not undergo rejection via CD47-SIRPα signaling.

### 5. SUMMARY

In one aspect, provided herein is a recombinant miniature swine, wherein the recombinant miniature swine comprises: (a) (i) a deleted or functionally inactivated endogenous gene encoding porcine CD47, wherein expression of the endogenous gene encoding porcine CD47 is regulated by porcine CD47 regulatory elements; and (ii) a transgene encoding human CD47 inserted within porcine genome, wherein expression of the transgene encoding human CD47 is regulated by the porcine CD47 regulatory elements; and (b) (i) a deleted or functionally inactivated endogenous gene encoding porcine SIRPα, wherein expression of the endogenous gene encoding porcine SIRPα is regulated by porcine SIRPA regulatory elements; and (ii) a transgene encoding human SIRPα inserted within porcine genome, wherein expression of the transgene encoding human SIRPα is regulated by the porcine SIRPA regulatory elements.

In some embodiments, the miniature swine is an alpha-1,3 galactosyltransferase-deficient miniature swine.

In some embodiments, the alpha-1,3 galactosyltransferase-deficient miniature swine is a major histocompatibility complex (MHC)-inbred miniature swine.

In some embodiments, the human CD47 comprises SEQ ID NO. 1, 2, or 3. In some embodiments, the human SIRPA comprises SEQ ID NO. 4 or 5.

In some embodiments, the transgenes encoding human SIRPα and human CD47 are inserted in the genome of the miniature swine by homologous recombination. In some embodiments, the transgenes encoding human SIRPα and human CD47 are inserted in the porcine genome by non-homology-directed end-joining. In some embodiments, the transgenes encoding human SIRPα and human CD47 are inserted in the porcine genome by recombinase-mediated cassette exchange. In some embodiments, the transgenes encoding human SIRPα and human CD47 are inserted in the porcine genome by a site-specific nuclease. In some embodiments, the site-specific nuclease is selected from the group consisting of zinc fingers, a ZFN dimer, a ZFNickase, transcription activator-like effector nucleases (TALENs), and CRISPR/Cas9.

In some embodiments, the expression of the transgene encoding human CD47 is regulated by the porcine CD47 regulatory elements of NCBI Gene ID: 397042. The recombinant miniature swine of claim 1, wherein expression of the transgene encoding human SIRPα is regulated by the porcine SIRPA regulatory elements of NCBI Gene ID: 494566.

In some embodiments, the human CD47 protein expression is substantially similar to an expression pattern of endogenous porcine CD47, as determined by immunohistochemistry. In some embodiments, the human SIRPα protein expression is substantially similar to an expression pattern of endogenous porcine SIRPα, as determined by immunohistochemistry.

In another aspect, provided herein is a recombinant miniature swine comprising a humanized CD47 gene; and/or (b) a humanized SIRPA gene. In some embodiments, the recombinant miniature swine comprises a humanized CD47 gene. In some embodiments, the recombinant miniature swine comprises a humanized SIRPA gene. In some embodiments, the recombinant miniature swine comprises a humanized CD47 gene and a humanized SIRPA gene. In some embodiments, the humanized CD47 gene comprises a porcine CD47 gene comprising exon 2 of human CD47.

In another aspect, provided herein is a cell derived from the recombinant miniature swine provided herein.

In another aspect, provided herein is an oocyte derived from the recombinant miniature swine provided herein.

In another aspect, provided herein is a sperm derived from the recombinant miniature swine provided herein.

In another aspect, provided herein is a tissue derived from the recombinant miniature swine provided herein.

In another aspect, provided herein is an organ derived from the recombinant miniature swine provided herein.

In yet another aspect, provided herein is a method of transplanting a graft from a first recombinant miniature swine to a primate, wherein the method comprises: (a) obtaining the graft from the first recombinant miniature swine provided herein; and (b) transplanting the graft to the primate. In some embodiments, the primate is a human. In some embodiments, the graft comprises cells, a tissue, or an organ. In some embodiments, the organ is selected from the group consisting of a heart, a kidney, an islet, a liver, a pancreas, a lung, an intestine, skin, a trachea, and a cornea, or a combination thereof.

In some embodiments, the method further comprises obtaining bone marrow from a second recombinant miniature swine provided herein, and transplanting the bone marrow to the same primate. In some embodiments, the bone marrow is transplanted at least 28 days before the graft from the first recombinant swine.

In some embodiments, the first recombinant miniature swine and the second recombinant miniature swine are the same recombinant swine. In some embodiments, the first recombinant miniature swine and the second recombinant miniature swine are from highly inbred herd of miniature swine. In some embodiments, the first recombinant miniature swine and the second recombinant miniature swine are genetically matched miniature swine. In some embodiments, the first recombinant miniature swine and the second recombinant miniature swine are MHC matched.

In some embodiments, the graft from the first recombinant miniature swine survives in the recipient at least for 6 months, 1 year, 5 years, 10 years, 15 years, or 20 years. In some embodiments, the graft from the first recombinant miniature swine functions in the recipient at least for 6 months, 1 year, 5 years, 10 years, 15 years, or 20 years.

In some embodiments, immunosuppressive therapies that are needed in the recipient are reduced by 90%, 80%, 70%, 60%, or 50%.

### 6. BRIEF DESCRIPTION OF THE DRAWINGS

FIG. 1A-FIG. 1D illustrate porcine CD47 expression levels following no guide pars (FIG. 1A), guide pair #1 (FIG. 1), guide pair #2 (FIG. 1C) or guide pair #3 (FIG. 1D).

FIG. 2 illustrates CD47 staining results for transfected cells sorted based on binding to anti-CD47 monoclonal antibody B6H12 (human only). Staining with anti-CD47 monoclonal antibody CC26, which binds both human and pig CD47, when blocked with human specific monoclonal B6H12 further demonstrated that a very high percentage of the population was null for porcine CD47.

### 7. DETAILED DESCRIPTION

Provided herein are recombinant miniature swine that express human or humanized CD47 and human or humanized SIRPA, but do not express endogenous porcine CD47 or porcine SIRPA. In specific aspects, expression of human or humanized CD47 and/or human or humanized SIRPA is under the same regulatory elements as the respective endogenous porcine gene. Generation of the recombinant miniature swine is described in Section 7.1. Also provided are cells, tissues, and organs derived from such recombinant miniature swine, which is described in Section 7.2. Furthermore, provided herein are methods of transplanting a graft from a first donor of such recombinant miniature swine with or without bone marrow from a second donor of such recombinant miniature swine, which is described in Section 7.3.

### 7.1 Generation of Recombinant Miniature Swine

Provided herein are genetically modified swine that express human or humanized CD47 and human or humanized SIRPA, and do not express endogenous porcine CD47 or porcine SIRPA. In specific aspects, expression of human or humanized CD47 and/or human or humanized SIRPA in the genetically modified swine is regulated by the endogenous regulatory elements (i.e., the procine porcine CD47 and porcine SIRPA regulatory elements, respectively). The cells, tissues, and organs of such genetically modified swine can be used for transplantation into a primate. Without being bound by any particular theory, such transplant not only ensures the health of donor swine and the graft to be transplanted, but also maximizes the long-term survival rate of the graft after transplanted into a primate.

Deleting or inactivating endogenous porcine CD47 and SIRPA and inserting the human homologs or portion thereof can be achieved using various methods known in the art, such as the methods described in Section 7.1.1 or the Examples. In certain embodiments, modifying the swine genome to delete the endogenous porcine CD47 and SIRPA genes or portion thereof, and inserting the human CD47 and SIRPA genes (such as the human homologs described in Section 7.1.2) or portion thereof can be performed in a single step. In certain embodiments, modifying the swine genome to delete the endogenous porcine CD47 and SIRPA genes or portion thereof and inserting the human CD47 and SIRPA genes or portion thereof can be performed in a two or more steps. A table of sequences of human CD47 and SIRPA to be inserted can be found in Table 1. In some embodiments, exon 2 of porcine CD47 is replaced with exon 2 of human CD47. Methods of experimentally determining the successful generation of the recombinant swine are described in Section 7.1.3. In addition, the donor miniature swine can carry additional genetic modifications, such as those described in Section 7.1.4.

In a specific embodiment, the donor miniature swine carries human CD47 and SIRPA transgenes that are expressed only when the endogenous CD47 and SIRPA genes of the donor animal are knocked out. In some embodiments, the endogenous CD47 and SIRPA genes of the donor animal are knocked out shortly before a graft is harvested for transplantation. This “genetic switch” allows the donor miniature swine to remain healthy during the majority of its lifetime, while still permitting expression of the human transgene for optimal transplant tolerance in a human recipient.

In a specific embodiment, the donor miniature swine expresses CD47 and SIRPA at levels that are comparable to human physiological levels of CD47 and SIRPA. For example, the levels of CD47 and SIRPα protein expressed in the kidney of a donor miniature swine are comparable to those levels of CD47 and SIRPα protein expressed in the kidney of a healthy human subject. Protein expression may be determined by a method known in the art or described herein.

In a specific embodiment, only one kidney of the miniature donor swine expresses CD47 and SIRPα protein.

### 7.1.1 Deleting Endogenous Porcine Genes and Inserting the Human Homologs

### 7.1.1.1 Deleting or Inactivating Endogenous Porcine Genes

In one aspect, provided herein is a method of deleting or inactivating endogenous porcine CD47 and SIRPA, and inserting human or humanized CD47 and human or humanized SIRPA.

Homologous recombination (HR) is a genetic recombination in which nucleotide sequences are exchanged between two similar or identical DNA molecules. In some embodiments, endogenous porcine CD47 and SIRPA are deleted or inactivated with HR-based methods to replace parts of the endogenous porcine CD47 and SIRPA. In some specific embodiments, the replacement of parts of the endogenous porcine CD47 and SIRPA leads to destruction of the start codons of the endogenous porcine CD47 and SIRPA. In other specific embodiments, the replacement of parts of the endogenous porcine CD47 and SIRPA leads to a destruction of the open reading frame. In other specific embodiments, the replacement of parts of the endogenous porcine CD47 and SIRPA blocks, prevents, or reduces transcription to negligible levels. In other specific embodiments, the replacement of part of the endogenous porcine CD47 and SIRPA leads to no translation products or non-functional translation products. In some embodiments, endogenous porcine CD47 and SIRPA is deleted or inactivated with HR-based methods to replace the entire full-length endogenous porcine CD47 and SIRPA with non-CD47 and non-SIRPA genes. In some embodiments, a portion (e.g., one or more exons) or porcine CD47 and/or SIRPA is replaced with a homologous region of human CD47 and/or SIRPA, respectively. In some specific embodiments, the non-CD47 and non-SIRPA genes are selection genes. In particular embodiments, non-CD47 and non-SIRPA genes are drug resistance genes.

In specific embodiments, the open reading frames of porcine CD47 and SIRPA are deleted and replaced with the open reading frames of human CD47 and SIRPA. In specific embodiments, the exons of porcine CD47 and SIRPA are deleted and replaced with the exons of human CD47 and SIRPA.

Without wishing to be bound by theory, deletion of the open reading frames of porcine CD47 and SIRPA, or of the exons of porcine CD47 and SIRPA, and replacing them with their respective human counterparts results in expression of the human CD47 and SIRPA under the control of the porcine regulatory elements of CD47 and SIRPA, respectively. The regulation of human CD47 and SIRPA under control of the porcine regulatory elements of the respective porcine genes results in similar expression patterns and levels of the human CD47 and SIRPA to those of the endogenous porcine CD47 and SIRPA. Gene expression levels may be determined using any suitable method known in the art, including, for example, quantitative PCR, real-time PCR, Southern Blotting or RNA sequencing.

In some embodiments, the expression level of human or humanized CD47 under the control of porcine regulatory elements is substantially similar to the expression levels of endogenous porcine CD47 (e.g., not more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% higher or lower than the expression of endogenous porcine CD47). In some embodiments, the expression level of human or humanized SIRPA under the control of porcine regulatory elements is substantially similar to the expression levels of endogenous porcine SIRPA (e.g., not more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% higher or lower than the expression of endogenous porcine SIRPA). In some embodiments, the expression level of both human or humanized CD47 and human or humanized SIRPA under the control of porcine regulatory elements is substantially similar to the expression levels of endogenous porcine CD47 and endogenous porcine SIRPA, respectively (e.g., not more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% higher or lower than the expression of endogenous porcine CD47 and endogenous porcine SIRPA).

In some embodiments, the spatial expression pattern of human or humanized CD47 under the control of porcine regulatory elements is substantially similar to the spatial expression pattern of endogenous porcine CD47. In some embodiments, the spatial expression pattern of human or humanized SIRPA under the control of porcine regulatory elements is substantially similar to the spatial expression pattern of endogenous porcine SIRPA. In some embodiments, the spatial expression pattern of both human or humanized CD47 and human or humanized SIRPA under the control of porcine regulatory elements is substantially similar to the spatial expression pattern of endogenous porcine CD47 and endogenous porcine SIRPA, respectively.

Spatial gene expression patterns may be determined using any suitable method known in the art, including, for example, single-cell sequencing, single-molecular fluorescence in situ hybridization, or spatial transcriptomics. Exemplary methods to analyze special gene expression patterns have been described, see, e.g., Sun et al., Nat Methods. 2020 February; 17(2):193-200 and Dries et al., Genome Biology (2021) 22:78.

In some embodiments, the temporal expression pattern of human or humanized CD47 under the control of porcine regulatory elements is substantially similar to the temporal expression pattern of endogenous porcine CD47 (e.g., no more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% delayed or accelerated compared to the expression of endogenous porcine CD47). In some embodiments, the temporal expression pattern of human or humanized SIRPA under the control of porcine regulatory elements is substantially similar to the temporal expression pattern of endogenous porcine SIRPA (e.g., no more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% delayed or accelerated compared to the expression of endogenous porcine SIRPA). In some embodiments, the temporal expression pattern of both human or humanized CD47 and human or humanized SIRPA under the control of porcine regulatory elements is substantially similar to the temporal expression pattern of endogenous porcine CD47 and endogenous porcine SIRPA, respectively (e.g., no more than about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% delayed or accelerated compared to the expression of endogenous porcine CD47 and endogenous porcine SIRPA).

Recombinase is an enzyme that recognizes specific polynucleotide sequences (recombinase recognition sites) that flank an intervening polynucleotide and catalyzes a reciprocal strand exchange, resulting in inversion or excision of the intervening polynucleotide. See, e.g., Araki et al. (1995), Proc. Natl. Acad. Sci. USA 92:160-164. In some embodiments, endogenous porcine CD47 and SIRPA are deleted or inactivated with recombinase-based methods and the recombinase recognition sites in the same orientation. In some specific embodiments, the locations of the recombinase recognition sites in the same orientation are designed so that the entire full length porcine genes are deleted. In some specific embodiments, the locations of the recombinase recognition sites in the same orientation are designed so that the parts of the porcine genes are deleted which leads to abnormal transcription or translation. In other embodiments, endogenous porcine CD47 and SIRPA are deleted or inactivated with recombinase-based methods the recombinase recognition sites in the opposite orientation. In some specific embodiments, the locations of the recombinase recognition sites in the opposite orientation are designed so that the entire full length porcine genes are reversed which leads to abnormal transcription or translation. In some specific embodiments, the locations of the recombinase recognition sites in the opposite orientation are designed so that the parts of the porcine genes are reversed which leads to abnormal transcription or translation.

Sequence-specific endonucleases includes, but are not limited to, RNA-guided DNA nucleases (e.g., the CRISPR/Cas9 system), a ZFN, a ZFN dimer, a ZFNickase, and TALENS. In some embodiments, endogenous porcine CD47 and SIRPA are deleted or inactivated with sequence-specific endonucleases-based methods. In some specific embodiments, the sequence-specific endonucleases are designed to target to destroy the start codons of the endogenous porcine CD47 and SIRPA. In other specific embodiments, the sequence-specific endonucleases are designed to target to destroy normal open reading frame of the endogenous porcine CD47 and SIRPA. In other specific embodiments, the sequence-specific endonucleases are designed to target to result in no transcription products. In yet other specific embodiments, the sequence-specific endonucleases are designed to target to result in no translation products or non-functional translation products.

### 7.1.1.2 Human Genes are Inserted into the Porcine Genome

In some embodiments, the transgene to be used to substitute porcine CD47 is the genomic sequence of human CD47 or a portion thereof. By using genomic DNA, all human CD47 isoforms can be expressed. In other embodiments, the transgene to be used to substitute porcine CD47 is the complementary DNA of message RNA (mRNA) for a known splice variant of human CD47. In other embodiments, the transgene to be used to substitute porcine CD47 is the coding sequence (CDS) of a known splice variant of human CD47. Nucleic acid sequences encoding human CD47 can be found under the following NCBI RefSeq accession numbers: NC_000003.12. cDNA of mRNA encoding human CD47 isoform 1 can be found under the following NCBI RefSeq accession numbers: NM_001777.4. cDNA of mRNA encoding human CD47 isoform 2 can be found under the following NCBI RefSeq accession numbers: NM_198793.3. cDNA of mRNA encoding human CD47 isoform 3 can be found under the following NCBI RefSeq accession numbers: NM_001382306.1. The CDS encoding human CD47 isoform 1 can be found under the following NCBI RefSeq accession numbers: CCDS43126.1 or SEQ ID No. 1, which corresponds to the amino acid sequence NP_001768.1. The CDS encoding human CD47 isoform 2 can be found under the following NCBI RefSeq accession numbers: CCDS43125.1 or SEQ ID No. 2, which corresponds to the amino acid sequence NP_942088.1. The CDS encoding human CD47 isoform 3 can be found under the following NCBI RefSeq accession numbers: SEQ ID No. 3, which corresponds to the amino acid sequence NP_001369235.1. In some embodiments, the transgene to be used to substitute porcine CD47 is one or more human exons (e.g., exon 2 of human CD47).

In certain embodiments, the transgene encoding human CD47 used in a construct described herein is a transgene listed in Table 1 below. In certain embodiments, the transgene encoding human CD47 comprises a nucleotide sequence of SEQ ID NO:1. In other embodiments, the transgene encoding human CD47 comprises a nucleotide sequence of SEQ ID NO:2. In other embodiments, the transgene encoding human CD47 comprises a nucleotide sequence of SEQ ID NO:3. In certain embodiments, the transgene encoding human CD47 comprises a nucleotide sequence that is at least 70%, at least 75%, at least 80%, at least 85%, at least 90%, at least 95% or at least 98% identical to SEQ ID NO:1. In other embodiments, the transgene encoding human CD47 comprises a nucleotide sequence that is at least 70%, at least 75%, at least 80%, at least 85%, at least 90%, at least 95% or at least 98% identical to SEQ ID NO:2. In other embodiments, the transgene encoding human CD47 comprises a nucleotide sequence that is at least 70%, at least 75%, at least 80%, at least 85%, at least 90%, at least 95% or at least 98% identical to SEQ ID NO:3.

In some embodiments, the transgene is the genomic sequence of human CD47, and the transgene replaces the genomic sequence of porcine CD47 expanding from the first to the last exons. In other embodiments, the transgene is the cDNA of human CD47, and the transgene replaces the genomic sequence of porcine CD47 expanding from the first to the last exons. In other embodiments, the transgene is the cDNA of human CD47, and the transgene replaces the porcine CD47 with porcine 5′ UTR and 3′ UTR intact. In yet other embodiments, the transgene is the CDS of human CD47, and the transgene replaces the porcine CD47 with porcine 5′ UTR and 3′ UTR intact. In some embodiments, the transgene is a portion of human CD47 (e.g., exon 2 of human CD47).

As provided herein, in certain aspects the CD47 transgene is a humanized CD47. In some embodiments, the humanized CD47 is endogenous porcine CD47 comprising one or more exons replaced with the corresponding one or more homologous exons of human CD47. For example, in some embodiments, the CD47 transgene is a humanized CD47 in which exon 1 of porcine CD47 is replaced with exon 1 of human CD47. In some embodiments, the CD47 transgene is a humanized CD47 in which exon 2 of porcine CD47 is replaced with exon 2 of human CD47. In some embodiments, the CD47 transgene is a humanized CD47 in which exon 3 of porcine CD47 is replaced with exon 3 of human CD47. In some embodiments, the CD47 transgene is a humanized CD47 in which exon 4 of porcine CD47 is replaced with exon 4 of human CD47. In some embodiments, the CD47 transgene is a humanized CD47 in which exon 5 of porcine CD47 is replaced with exon 5 of human CD47. In some embodiments, the CD47 transgene is a humanized CD47 in which exon 6 of porcine CD47 is replaced with exon 6 of human CD47. In some embodiments, the CD47 transgene is a humanized CD47 in which exon 7 of porcine CD47 is replaced with exon 7 of human CD47. In some embodiments, the CD47 transgene is a humanized CD47 in which exon 8 of porcine CD47 is replaced with exon 8 of human CD47. In some embodiments, the CD47 transgene is a humanized CD47 in which exon 9 of porcine CD47 is replaced with exon 9 of human CD47. In some embodiments, the CD47 transgene is a humanized CD47 in which exon 10 of porcine CD47 is replaced with exon 10 of human CD47. In some embodiments, a portion of the coding sequence of porcine CD47 is replaced with a portion of the coding sequence of human CD47. In some embodiments, about 10% of the coding sequence of porcine CD47 is replaced with about 10% of the coding sequence of the corresponding homologous portion of human CD47. In some embodiments, a portion of the coding sequence of porcine CD47 is replaced with a portion of the coding sequence of human CD47. In some embodiments, about 20% of the coding sequence of porcine CD47 is replaced with about 20% of the coding sequence of the corresponding homologous portion of human CD47. In some embodiments, a portion of the coding sequence of porcine CD47 is replaced with a portion of the coding sequence of human CD47. In some embodiments, about 30% of the coding sequence of porcine CD47 is replaced with about 30% of the coding sequence of the corresponding homologous portion of human CD47. In some embodiments, a portion of the coding sequence of porcine CD47 is replaced with a portion of the coding sequence of human CD47. In some embodiments, about 40% of the coding sequence of porcine CD47 is replaced with about 40% of the coding sequence of the corresponding homologous portion of human CD47. In some embodiments, a portion of the coding sequence of porcine CD47 is replaced with a portion of the coding sequence of human CD47. In some embodiments, about 50% of the coding sequence of porcine CD47 is replaced with about 50% of the coding sequence of the corresponding homologous portion of human CD47. In some embodiments, a portion of the coding sequence of porcine CD47 is replaced with a portion of the coding sequence of human CD47. In some embodiments, about 60% of the coding sequence of porcine CD47 is replaced with about 60% of the coding sequence of the corresponding homologous portion of human CD47. In some embodiments, a portion of the coding sequence of porcine CD47 is replaced with a portion of the coding sequence of human CD47. In some embodiments, about 70% of the coding sequence of porcine CD47 is replaced with about 70% of the coding sequence of the corresponding homologous portion of human CD47. In some embodiments, a portion of the coding sequence of porcine CD47 is replaced with a portion of the coding sequence of human CD47. In some embodiments, about 80% of the coding sequence of porcine CD47 is replaced with about 80% of the coding sequence of the corresponding homologous portion of human CD47. In some embodiments, a portion of the coding sequence of porcine CD47 is replaced with a portion of the coding sequence of human CD47. In some embodiments, about 90% of the coding sequence of porcine CD47 is replaced with about 90% of the coding sequence of the corresponding homologous portion of human CD47. In some embodiments, a portion of the coding sequence of porcine CD47 is replaced with a portion of the coding sequence of human CD47. In some embodiments, about 95% of the coding sequence of porcine CD47 is replaced with about 95% of the coding sequence of the corresponding homologous portion of human CD47.

In some embodiments, the transgene used to substitute porcine SIRPA is the genomic sequence of human SIRPA. By using genomic DNA, all human SIRPA isoforms can be expressed. In other embodiments, the transgene to be used to substitute porcine SIRPA is the complementary DNA of message RNA (mRNA) for a known splice variant of human SIRPA. In other embodiments, the transgene to be used to substitute porcine SIRPA is the coding sequence (CDS) of a known splice variant of human SIRPA. Nucleic acid sequences encoding human SIRPα can be found under the following NCBI RefSeq accession numbers: NC_000020.11. cDNA of mRNA encoding human SIRPα isoform 1 can be found under the following NCBI RefSeq accession numbers: NM_080792.3, NM_001040022.1 or NM_001040023.2. cDNA of mRNA encoding human SIRPα isoform 2 can be found under the following NCBI RefSeq accession numbers: NM_001330728.1. The CDS encoding human SIRPα isoform 1 can be found under the following NCBI RefSeq accession numbers: CCDS13022.1 or SEQ ID NO. 4, which corresponds to the amino acid sequence NP_001035111.1, NP_001035112.1, or NP_542970.1. The CDS encoding human SIRPα isoform 2 can be found under the following NCBI RefSeq accession numbers: CCDS82593.1 or SEQ ID NO. 5, which corresponds to the amino acid sequence NP_001317657.1. In some embodiments, the transgene is a portion of human SIRPA.

In certain embodiments, the transgene encoding human SIRPα used in a construct described herein is a transgene listed in Table 1 below. In certain embodiments, the transgene encoding human SIRPα comprises a nucleotide sequence of SEQ ID NO:4. In other embodiments, the transgene encoding human SIRPα comprises a nucleotide sequence of SEQ ID NO:5. In certain embodiments, the transgene encoding human SIRPα comprises a nucleotide sequence that is at least 70%, at least 75%, at least 80%, at least 85%, at least 90%, at least 95% or at least 98% identical to SEQ ID NO:4. In other embodiments, the transgene encoding human SIRPα comprises a nucleotide sequence that is at least 70%, at least 75%, at least 80%, at least 85%, at least 90%, at least 95% or at least 98% identical to SEQ ID NO:5.

In some embodiments, if the transgene is the genomic sequence of human SIRPA, the transgene replaces the genomic sequence of porcine SIRPA expanding from the first to the last exons. In other embodiments, if the transgene is the cDNA of human SIRPA, the transgene replaces the genomic sequence of porcine SIRPA expanding from the first to the last exons. In other embodiments, if the transgene is the cDNA of human SIRPA, the transgene replaces the porcine SIRPA with porcine 5′ UTR and 3′ UTR intact. In yet other embodiments, if the transgene is the CDS of human SIRPA, the transgene replaces the porcine SIRPA with porcine 5′ UTR and 3′ UTR intact. In some embodiments, the transgene replaces a homolgous exon of porcine SIRPA (e.g., exon 2 of human SIRPA replaces exon 2 of procine SIRPA).

As provided herein, in certain aspects the SIRPA transgene is a humanized SIRPA. In some embodiments, the humanized SIRPA is endogenous porcine SIRPA comprising one or more exons replaced with the corresponding one or more homologous exon of human SIRPA. For example, in some embodiments, the SIRPA transgene is a humanized SIRPA in which exon 1 of porcine SIRPA is replaced with exon 1 of human SIRPA. For example, in some embodiments, the SIRPA transgene is a humanized SIRPA in which exon 2 of porcine SIRPA is replaced with exon 2 of human SIRPA. For example, in some embodiments, the SIRPA transgene is a humanized SIRPA in which exon 3 of porcine SIRPA is replaced with exon 3 of human SIRPA. For example, in some embodiments, the SIRPA transgene is a humanized SIRPA in which exon 4 of porcine SIRPA is replaced with exon 4 of human SIRPA. For example, in some embodiments, the SIRPA transgene is a humanized SIRPA in which exon 5 of porcine SIRPA is replaced with exon 5 of human SIRPA. For example, in some embodiments, the SIRPA transgene is a humanized SIRPA in which exon 6 of porcine SIRPA is replaced with exon 6 of human SIRPA. For example, in some embodiments, the SIRPA transgene is a humanized SIRPA in which exon 7 of porcine SIRPA is replaced with exon 7 of human SIRPA. For example, in some embodiments, the SIRPA transgene is a humanized SIRPA in which exon 8 of porcine SIRPA is replaced with exon 8 of human SIRPA. For example, in some embodiments, the SIRPA transgene is a humanized SIRPA in which exon 9 of porcine SIRPA is replaced with exon 9 of human SIRPA. For example, in some embodiments, the SIRPA transgene is a humanized SIRPA in which exon 10 of porcine SIRPA is replaced with exon 10 of human SIRPA. For example, in some embodiments, the SIRPA transgene is a humanized SIRPA in which exon 11 of porcine SIRPA is replaced with exon 11 of human SIRPA. For example, in some embodiments, the SIRPA transgene is a humanized SIRPA in which exon 12 of porcine SIRPA is replaced with exon 12 of human SIRPA. In some embodiments, a portion of the coding sequence of porcine SIRPA is replaced with a portion of the coding sequence of human SIRPA. In some embodiments, about 10% of the coding sequence of porcine SIRPA is replaced with about 10% of the coding sequence of the corresponding homologous portion of human SIRPA. In some embodiments, a portion of the coding sequence of porcine SIRPA is replaced with a portion of the coding sequence of human SIRPA. In some embodiments, about 20% of the coding sequence of porcine SIRPA is replaced with about 20% of the coding sequence of the corresponding homologous portion of human SIRPA. In some embodiments, a portion of the coding sequence of porcine SIRPA is replaced with a portion of the coding sequence of human SIRPA. In some embodiments, about 30% of the coding sequence of porcine SIRPA is replaced with about 30% of the coding sequence of the corresponding homologous portion of human SIRPA. In some embodiments, a portion of the coding sequence of porcine SIRPA is replaced with a portion of the coding sequence of human SIRPA. In some embodiments, about 40% of the coding sequence of porcine SIRPA is replaced with about 40% of the coding sequence of the corresponding homologous portion of human SIRPA. In some embodiments, a portion of the coding sequence of porcine SIRPA is replaced with a portion of the coding sequence of human SIRPA. In some embodiments, about 50% of the coding sequence of porcine SIRPA is replaced with about 50% of the coding sequence of the corresponding homologous portion of human SIRPA. In some embodiments, a portion of the coding sequence of porcine SIRPA is replaced with a portion of the coding sequence of human SIRPA. In some embodiments, about 60% of the coding sequence of porcine SIRPA is replaced with about 60% of the coding sequence of the corresponding homologous portion of human SIRPA. In some embodiments, a portion of the coding sequence of porcine SIRPA is replaced with a portion of the coding sequence of human SIRPA. In some embodiments, about 70% of the coding sequence of porcine SIRPA is replaced with about 70% of the coding sequence of the corresponding homologous portion of human SIRPA. In some embodiments, a portion of the coding sequence of porcine SIRPA is replaced with a portion of the coding sequence of human SIRPA. In some embodiments, about 80% of the coding sequence of porcine SIRPA is replaced with about 80% of the coding sequence of the corresponding homologous portion of human SIRPA. In some embodiments, a portion of the coding sequence of porcine SIRPA is replaced with a portion of the coding sequence of human SIRPA. In some embodiments, about 90% of the coding sequence of porcine SIRPA is replaced with about 90% of the coding sequence of the corresponding homologous portion of human SIRPA. In some embodiments, a portion of the coding sequence of porcine SIRPA is replaced with a portion of the coding sequence of human SIRPA. In some embodiments, about 95% of the coding sequence of porcine SIRPA is replaced with about 95% of the coding sequence of the corresponding homologous portion of human SIRPA.

Expression cassettes generally comprise a regulatory element and a transgene. A regulatory element may be, for example, a promoter. In some embodiments, the promoter for the human or humanized CD47 is a constitutively active promoter. In other embodiments, the promoter for the human or humanized CD47 is the endogenous porcine CD47 promoter.

In some embodiments, the human CD47 and SIRPA transgene are inserted randomly into a locus in the porcine genome. In some embodiments, the human CD47 and SIRPA transgene are inserted into a safe harbor locus in the porcine genome. In some embodiments, the human CD47 transgene is inserted near porcine CD47 gene (NCBI Gene ID: 397042) and the porcine CD47 gene is not deleted. In some embodiments, the human CD47 transgene is inserted in the endogenous porcine CD47 gene and the porcine CD47 gene is deleted. In some embodiments, the human SIRPA transgene is inserted near porcine SIRPA gene (NCBI Gene ID: 494566) and the porcine SIRPA gene is not deleted. In some embodiments, the human SIRPA transgene is inserted in the endogenous porcine SIRPA gene and the porcine SIRPA gene is deleted. In some embodiments, a portion of human CD47 or SIRPA replaces the homologous exon of porcine CD47 or SIPRA, respectively.

### 7.1.1.3 Delivery Vehicles and Methods

In some embodiments, the plasmids or vectors that carry the components described in Sections 7.1.1.1 and 7.1.1.2 are delivered in viral vectors, which include, but are not limited to, adeno-associated virus (AAV), self-complimentary adeno-associated virus (scAAV), adenovirus, retrovirus, lentivirus (e.g., Simian immunodeficiency virus, human immunodeficiency virus, or modified human immunodeficiency virus), Newcastle disease virus (NDV), herpes virus (e.g., herpes simplex virus), alphavirus, vaccina virus. Viral vectors may further comprise other elements, such as a Poly(A) site, a transcription termination site, or viral-specific elements such as inverted terminal repeats. See, e.g. Buard et al. (2009), British Journal of Pharmacology 157:153-165. In other embodiments, some of the plasmids or vectors that carry the components described in Sections 7.1.1.1 and 7.1.1.2 are delivered via transposases, which include, but are not limited to, sleeping beauty and piggyback.

The methods of introducing the human or humanized CD47 and SIRPA genes to the germline of the animal includes, but are not limited to, somatic cell nuclear transfer (SCNT), pronuclear microinjection, sperm-mediated gene transfer (SMGT), oocyte transduction, and intracytoplasmic sperm injection-mediated transgenesis. See, e.g., Yum et al. (2016) J Vet Sci 2016, 17:261-268; Whyte and Prather (2011), Mol Reprod Dev 78:879-891; Sachs and Gali (2009). Pronuclear microinjection involves the direct injection of DNA into the pronuclei. Eggs for these purposes may be collected from a superovulated female, and then transferred to a recipient pig by embryo transfer. See, e.g., Whyte and Prather (2011), Mol Reprod Dev 78:879-891. SMGT involves incubating genes for the transgene of interest with spermatozoa, which are subsequently used for insemination. See, e.g., Lavitrano et al., (2002), Proc Nat Acad Sci USA. 99:14230-14235. Oocyte transduction includes maturing porcine oocytes in vitro in a serum-free, chemically defined maturation medium, which is subsequently infected with a replication deficient pseudotyped retrovirus, fertilized and cultured in vitro before being transferred to a recipient female. See, e.g., Cabot et al., (2001), Anim Biotechnol; 12(2):205-14. Intracytoplasmic sperm injection-mediated transgenesis encompasses in vitro matured porcine oocytes fertilized by intracytoplasmic sperm injection. See, e.g., Lai et al., (2001) Zygote; 9(4):339-46.

### 7.1.2 Substituting Endogenous Porcine Genes with the Human Homologs

Constructs for the expression of transgenes generally comprise a nucleotide sequence encoding the transgenes, e.g., human or humanized CD47 or SIRPα, as described in Section 7.1.2.1. Additionally, constructs for the expression of transgenes comprise other elements responsible for the chosen substitution technologies described in Section 7.1.2.2, and potential electable markers (positive and/or negative). With the constructs of Section 7.1.2.1 and strategies of substitution in Section 7.1.2.2, human or humanized CD47 is inserted under the same regulatory elements of NCBI Gene ID: 397042 (endogenous porcine CD47), and the human or humanized SIRPA is inserted under the same regulatory elements of NCBI Gene ID: 494566 (endogenous porcine SIRPA).

### 7.1.2.1 Sequences for Human CD47 and SIRPA

In some embodiments, the transgene to be used to substitute porcine CD47 or portion thereof is the genomic sequence of human CD47 or portion thereof. By using genomic DNA, all human CD47 isoforms can be expressed. In other embodiments, the transgene to be used to substitute porcine CD47 or portion thereof is the complementary DNA of message RNA (mRNA) or portion thereof for a known splice variant of human CD47. In other embodiments, the transgene to be used to substitute porcine CD47 or portion thereof is the coding sequence (CDS) or portion thereof of a known splice variant of human CD47. Nucleic acid sequences encoding human CD47 or portion thereof can be found under the following NCBI RefSeq accession numbers: NC_000003.12. cDNA of mRNA encoding human CD47 isoform 1 can be found under the following NCBI RefSeq accession numbers: NM_001777.4. cDNA of mRNA encoding human CD47 isoform 2 can be found under the following NCBI RefSeq accession numbers: NM_198793.3. cDNA of mRNA encoding human CD47 isoform 3 can be found under the following NCBI RefSeq accession numbers: NM_001382306.1. The CDS encoding human CD47 isoform 1 can be found under the following NCBI RefSeq accession numbers: CCDS43126.1 or SEQ ID No. 1, which corresponds to the amino acid sequence NP_001768.1. The CDS encoding human CD47 isoform 2 can be found under the following NCBI RefSeq accession numbers: CCDS43125.1 or SEQ ID No. 2, which corresponds to the amino acid sequence NP_942088.1. The CDS encoding human CD47 isoform 3 can be found under the following NCBI RefSeq accession numbers: SEQ ID No. 3, which corresponds to the amino acid sequence NP_001369235.1.

In certain embodiments, the transgene encoding human CD47 used in a construct described herein is a transgene listed in Table 1 below. In certain embodiments, the transgene encoding human CD47 comprises a nucleotide sequence of SEQ ID NO:1. In other embodiments, the transgene encoding human CD47 comprises a nucleotide sequence of SEQ ID NO:2. In other embodiments, the transgene encoding human CD47 comprises a nucleotide sequence of SEQ ID NO:3. In certain embodiments, the transgene encoding human CD47 comprises a nucleotide sequence that is at least 70%, at least 75%, at least 80%, at least 85%, at least 90%, at least 95% or at least 98% identical to SEQ ID NO:1. In some embodiments, the transgene encoding human CD47 comprises a nucleotide sequence that is at least 70%, at least 75%, at least 80%, at least 85%, at least 90%, at least 95% or at least 98% identical to SEQ ID NO:2. In some embodiments, the transgene encoding human CD47 comprises a nucleotide sequence that is at least 70%, at least 75%, at least 80%, at least 85%, at least 90%, at least 95% or at least 98% identical to SEQ ID NO:3.

In some embodiments, the transgene is the genomic sequence of human CD47, and the transgene replaces the genomic sequence of porcine CD47 expanding from the first to the last exons. In other embodiments, the transgene is the cDNA of human CD47, and the transgene replaces the genomic sequence of porcine CD47 expanding from the first to the last exons. In other embodiments, the transgene is the cDNA of human SIRPA, and the transgene replaces the porcine CD47 with porcine 5′ UTR and 3′ UTR intact. In yet other embodiments, the transgene is the CDS of human CD47, and the transgene replaces the porcine CD47 with porcine 5′ UTR and 3′ UTR intact. In some embodiments, the transgene replaces a portion of porcine CD47 (e.g., a single exon) with a portion of human CD47 (e.g., a homologous exon).

In other embodiments, the DNA sequence of the CD47 transgene is non-human. For example, the CD47 DNA sequence corresponds with a DNA sequence selected from a group of non-human CD47 sequences that includes NCBI Gene ID Numbers: 460569 (Pan troglodytes or chimpanzee), 704980 (Macaca mulatta or Rhesus monkey), 478552 (Canis lupusfamiliaris or dog), 282661 (Bos taurus or cattle), 16423 (Mus musculus or house mouse), 29364 (Rattus norvegicus or Norway rat), 418408 (Gallus or chicken), 100926819 (Sarcophilus harrisii or Tasmanian devil), 102089340 (Columba livia or rock pigeon), 101681023 (Mustela putorius furo or domestic ferret), 109691157 (Castor canadensis or American beaver), and 101836211 (Mesocricetus auratus or golden hamster).

In some embodiments, the transgene to be used to substitute porcine SIRPA or portion thereof is the genomic sequence of human SIRPA or portion thereof. By using genomic DNA, all human SIRPα isoforms can be expressed. In other embodiments, the transgene to be used to substitute porcine SIRPA or portion thereof is the complementary DNA of message RNA (mRNA) or portion thereof for a known splice variant of human SIRPα. In other embodiments, the transgene to be used to substitute porcine SIRPA or portion thereof is the coding sequence (CDS) or portion thereof of a known splice variant of human SIRPα. Nucleic acid sequences encoding human SIRPα can be found under the following NCBI RefSeq accession numbers: NC_000020.11. cDNA of mRNA encoding human SIRPα isoform 1 can be found under the following NCBI RefSeq accession numbers: NM_080792.3, NM_001040022.1 or NM_001040023.2. cDNA of mRNA encoding human SIRPα isoform 2 can be found under the following NCBI RefSeq accession numbers: NM_001330728.1. The CDS encoding human SIRPα isoform 1 can be found under the following NCBI RefSeq accession numbers: CCDS13022.1 or SEQ ID NO. 4, which corresponds to the amino acid sequence NP_001035111.1, NP_001035112.1, or NP_542970.1. The CDS encoding human SIRPα isoform 2 can be found under the following NCBI RefSeq accession numbers: CCDS82593.1 or SEQ ID NO. 5, which corresponds to the amino acid sequence NP_001317657.1.

In certain embodiments, the transgene encoding human SIRPα used in a construct described herein is a transgene listed in Table 1 below. In certain embodiments, the transgene encoding human SIRPα comprises a nucleotide sequence of SEQ ID NO:4. In other embodiments, the transgene encoding human SIRPα comprises a nucleotide sequence of SEQ ID NO:5. In certain embodiments, the transgene encoding human SIRPα comprises a nucleotide sequence that is at least 70%, at least 75%, at least 80%, at least 85%, at least 90%, at least 95% or at least 98% identical to SEQ ID NO:4. In other embodiments, the transgene encoding human SIRPα comprises a nucleotide sequence that is at least 70%, at least 75%, at least 80%, at least 85%, at least 90%, at least 95% or at least 98% identical to SEQ ID NO:5.

In some embodiments, the transgene is the genomic sequence of human SIRPA, and the transgene replaces the genomic sequence of porcine SIRPA expanding from the first to the last exons. In other embodiments, the transgene is the cDNA of human SIRPA, and the transgene replaces the genomic sequence of porcine SIRPA expanding from the first to the last exons. In other embodiments, the transgene is the cDNA of human SIRPA, and the transgene replaces the porcine SIRPA with porcine 5′ UTR and 3′ UTR intact. In yet other embodiments, the transgene is the CDS of human SIRPA, and the transgene replaces the porcine SIRPA with porcine 5′ UTR and 3′ UTR intact. In some embodiments, the transgene replaces a portion of porcine SIRPA (e.g., a single exon) with a portion of human SIRPA (e.g., a homologous exon).

In other embodiments, the DNA sequence of the SIRPA transgene is non-human. For example, the SIRPA DNA sequence corresponds with a DNA sequence selected from a group of non-human SIRPA sequences that includes NCBI Gene ID Numbers: 458039 (Pan troglodytes or chimpanzee), 717811 (Macaca mulatta or Thesus monkey), 101926317 (Macaca fascicularis or crab-eating macaque), 609452 (Canis lupusfamiliaris or dog), 327666 (Bos taurus or cattle), 19261 (Mus musculus or house mouse), 25528 (Rattus norvegicus or Norway rat), 118618252 (Molossus or Pallas's mastiff bat), 109692903 (Castor canadensis or American beaver), 101677644 (Mustela putorius furo or domestic ferret), 101839275 (Mesocricetus auratus or golden hamster), 100067339 (Equus caballus or horse), 118536470 (Halichoerus grypus or gray seal), 118011325 (Mirounga 23dminis or Southern elephant seal), 116739680 (Phocoena sinus or vaquita), 116641565 (Phoca vitulina or harbor seal), 116563017 (Sapajus apella or Tufted capuchin), or 116461231 (Hylobates moloch or silvery gibbon).

### 7.1.2.2 Strategies of Substitution

Gene replacement of porcine CD47 and SIRPA or portion thereof with human CD47 and SIRPA or portion thereof can be performed with various methods known in the art. For example, one non-limiting exemplary technique includes homolgous recombination. HR has been widely used by researchers in gene targeting and making of transgenic animals. HR is a genetic recombination in which nucleotide sequences are exchanged between two similar or identical DNA molecules. Gene targeting with homologous recombination in embryonic stem cells allows unprecedented precision with which one could manipulate genes and study the effect of this manipulation.

Another exemplary technique suitable for gene replacement of porcine CD47 and SIRPA or portion thereof with human CD47 and SIRPA or portion thereof, includes non-homology-directed end-joining (NHEJ). NHEJ directly ligates the ends of DSBs, and there is little or no homologous sequence in comparison with HR. It has been utilized by some researchers to integrate genes of interest with a concomitant usage of site-specific endonucleases with great success. See, e.g., Schiermeyer et al. (2019) Plant Direct.; 3(7): e00153.

Yet another exemplary technique suitable for gene replacement of porcine CD47 and SIRPA or portion thereof with human CD47 and SIRPA or portion thereof, includes recombinase-mediated cassette exchange (RMCE). RCME technology enables the swapping of large genomic regions. This technology is generally recommended for the generation of humanized models and several mutants using the same parental embryonic stem cell clone. RMCE technology allows the substitution of large genomic regions in the host genome with the concomitant human genomic regions, because RMCE can be easily adapted specifically for the integration of genomic DNAs.

In some embodiments, gene replacement of the porcine CD47 and SIRPA or portion thereof for human CD47 and SIRPA or portion thereof can be generated using an enzyme that promotes DNA DSBs. In some embodiments, the enzyme that promotes DNA DSBs includes a sequence-specific endonuclease. In specific embodiments, the sequence-specific endonuclease includes a RNA-guided DNA nucleases, e.g., the CRISPR/Cas9 system.

The CRISPR/Cas9 (Clustered Regularly-Interspaced Short Palindromic Repeats) system exploits RNA-guided DNA-binding and sequence-specific cleavage of target DNA. A guide RNA (gRNA) (e.g., containing 20 nucleotides) are complementary to a target genomic DNA sequence upstream of a genomic PAM (protospacer adjacent motifs) site (NNG) and a constant RNA scaffold region. The Cas (CRISPR-associated) protein binds to the gRNA and the target DNA to which the gRNA binds and introduces a double-strand break in a defined location upstream of the PAM site. See, e.g., Geurts et al. (2009), Science 325:433; Mashimo et al. (2010), PloS ONE 5, e8870; Carbery et al. (2010), Genetics 186:451-459; Tesson et al. (2011), Nat. Biotech. 29:695-696; Wiedenheft et al. (2012), Nature 482, 331-338; Jinek et al. (2012), Science 337:816-821; Mali et al. (2013), Science 339:823-826; Cong et al. (2013), Science 339:819-823. Further improvements of CRISPR/Cas9 system include using truncated gRNA with shorter regions of target complementary less than 20 nucleotides in length, can decrease undesired mutagenesis at off-target sites by as much as 5000-fold or more without sacrificing on-target genome editing efficiencies. See, e.g., Fu et al. (2014) Nat Biotechnol.; 32(3): 279-284. A modified CRISPR/Cas9-assisted HR uses a single-stranded oligodeoxynucleotide (ssODN) as a repair template (also known as “easiCRISPR”), is sufficient for efficient editing and replacement of host genes with human counterparts. Its advantages include no needs for selection and therefore without genomic scarring. See, e.g., Quadros et al. (2017) Genome. Biol. 18, 92; Codner et al. (2018) BMC Biol. 16,70.

In some embodiments, the sequence-specific endonucleases includes a ZFN, a ZFN dimer and/or a ZFNickase. ZFNs have separate DNA-binding and DNA-cleavage domains. The cleavage domains have no apparent sequence specificity, whose cutting could be redirected by the separate DNA-binding domains. See, e.g., Kim et al. (1994); Proc. Natl. Acad. Sci. USA 91: 883-887; Kim et al. (1996) Proc. Natl. Acad. Sci. USA 93: 1156-1160; Kim et al. (1998) Biol. Chem. 379: 489-495. Each unit of ˜30 amino acids of the zinc fingers (ZFs) binds to a single atom of zinc. The crystal structure of a set of three fingers bound to DNA showed that each finger contacts primarily 3 bp of DNA in remarkably modular fashion. See, e.g., Pavletich and Pabo (1991) Science 252: 809-817. This suggested that many different sequences could be attacked by making novel assemblies of ZFs. See, e.g., Carroll (2011), Genetics.; 188(4): 773-782. On the other hand, ZFNickases are created by inactivating the catalytic activity of one ZFN monomer in the ZFN dimer required for double-strand cleavage. ZFNickases demonstrate strand-specific nicking activity in vitro and thus provide for highly specific single-strand breaks in DNA. See, e.g., Ramirez et al. (2012); Nucleic Acids Research. 40 (7): 5560-5568.

In some embodiments, the sequence-specific endonucleases includes TALENS. TALENs comprise a non-specific DNA-cleaving nuclease fused to a DNA-binding domain that can be engineered so that TALENs can target essentially any sequence. The DNA-binding domain of TALENs is composed of highly conserved repeats derived from transcription activator-like effectors (TALEs). Researchers use a simple “protein-DNA code” that relates the DNA-binding TALE domains to individual bases in a target-binding site. Therefore, with its ease of design, high rates of cleavage activity, and the essentially limitless targeting range, TALENs has gained its popularity in introducing targeted DSBs and genome editing. See, e.g., Joung et al. (2013) Nat Rev Mol Cell Biol.; 14(1): 49-55.

In some embodiments, the sequence-specific insertion (or knock-in) of human CD47 and SIRPA transgene under the same regulatory elements as the endogenous porcine CD47 gene within the genome of the miniature swine may be achieved by HR, NHEJ, and/or RMCE. In preferred embodiments, the sequence-specific insertion (or knock-in) of human CD47 and SIRPA transgene under the same regulatory elements as the endogenous porcine CD47 gene within the genome of the miniature swine may be achieved by HR facilitated by sequence-specific endonucleases. In preferred embodiments, the sequence-specific insertion (or knock-in) of human CD47 and SIRPA transgene under the same regulatory elements as the endogenous porcine CD47 gene within the genome of the miniature swine may be achieved by NHEJ facilitated by sequence-specific endonucleases. In preferred embodiments, the sequence-specific insertion (or knock-in) of human CD47 and SIRPA transgene under the same regulatory elements as the endogenous porcine CD47 gene within the genome of the miniature swine may be achieved by RMCE in combination with sequence-specific endonucleases. See, e.g., Meyer et al. (2010), Proc. Natl. Acad. Sci. USA 107:15022-15026; Cui et al. (2010), Nat. Biotechnol. 29:64-67; Moehle et al. (2007), Proc Natl Acad Sci USA 104:3055-3060. This process relies on targeting specific gene sequences with endonucleases that recognize and bind to such sequences and induce a double-strand break in the nucleic acid molecule of the miniature swine cell. The double-strand break is then repaired. If, for example, a template (e.g., a construct containing the human CD47 and another construct containing the human SIRPA) is provided in trans, the double-strand break can be repaired using the provided template. Therefore, the expression cassettes containing human CD47 or SIRPA are integrated into the genome at specific loci. Non-limiting examples of the endonucleases include a zinc finger nuclease (ZFN), a ZFN dimer, a ZFNickase, a transcription activator-like effector nuclease (TALEN), or a RNA-guided DNA endonuclease (e.g., CRISPR/Cas9).

In some embodiments, one endonuclease is used, whose targeting site is upstream of the desired substitution region. In other embodiments, one endonuclease is used, whose targeting site is downstream of the desired substitution region. In other embodiments, one endonuclease is used, whose targeting site is within the desired substitution region. In some embodiments, two or more endonucleases are used, and the targeting sites of each endonuclease is upstream of the desired substitution region. In other embodiments, two or more endonucleases are used, and the targeting sites of each endonuclease is downstream of the desired substitution region. In other embodiments, two or more endonucleases are used, and the targeting sites of each endonuclease is within the desired substitution region. In yet other embodiments, two or more endonucleases are used, and at least one targeting site is upstream of the desired substitution region and at least one targeting site is downstream of the desired substitution. In some embodiments, two or more endonucleases are used, and at least one targeting site is upstream of the desired substitution region and at least one targeting site is within the desired substitution. In some embodiments, two or more endonucleases are used, and at least one targeting site is downstream of the desired substitution region and at least one targeting site is within the desired substitution. In some embodiments, three or more endonucleases are used, and at least one targeting site is upstream of the desired substitution region, at least one targeting site is within the desired substitution, and at least one targeting site is downstream of the desired substitution region.

In some embodiments, after preparing the plasmids and/or vectors containing the elements describing in the preceding paragraphs, the plasmids and/or vectors are transfected into cells, including but not limiting to, cultured porcine fetal fibroblasts. Transfection technologies include, but do not limit to, physical transfection methods (e.g., electroporation, direct injection, biolistic particle delivery, laser-irradiation, sonoporation, magnetic nanoparticle), chemical transfection methods (e.g., calcium phosphate, cationic polymer such as lipofectamine or lipofectin, cationic lipid), and biological transfection methods (e.g., virus-medicated delivery).

In some embodiments, after transfection of the cells, positive or/and negative selection is carried out according to the included selectable labels.

In certain aspects, provided herein are porcine cells comprising deletion or inactivation of endogenous porcine CD47 and SIRPA, and human CD47 and SIRPA transgenes suitable for use in generating recombinant miniature swine. Various methods of making transgenic animals (e.g., miniature swine) are known in the art See, e.g., Hryhorowicz et al. (2020), Genes 2020, 11, 670. Non-limiting exemplary methods are described herein below.

In certain embodiments, miniature swine from an inbred herd of miniature swine are used. The transgenic animal may be produced by any suitable method known in the art. For example, the gene expression construct may be introduced into the germline of the animal using, such as SCNT. SCNT involves the transfer of the nucleus of a donor cell into an oocyte or early embryo from which the chromosomes have been removed. See, e.g., Wilmut and Taylor (2015), Phil. Trans. R. Soc. B 370:20140366. Reconstructed embryos are surgically transferred into the oviduct of a surrogate pig in estrus. The recombinant miniature swine is delivered by the surrogate sows. Method of testing and verifying expression of the transgene in the transgenic pigs is discussed in section 7.1.3.

In specific embodiments, a plasmid or a vector containing a human CD47 sequence flanked by two homology arms, one of which is upstream the porcine CD47 gene sequence and the other of which is the downstream sequence of porcine CD47 gene, is generated. In some embodiments, a plasmid or a vector containing a portion of a human CD47 sequence flanked by two homology arms, one of which is upstream the target portion of the porcine CD47 gene sequence and the other of which is the downstream the target portion of porcine CD47 gene, is generated. One or more selectable marker are included in the plasmid or the vector. The above-described plasmid or vector is transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a plasmid or a vector containing a human CD47 sequence flanked by two homology arms, one of which is upstream the porcine CD47 gene sequence and the other of which is the downstream sequence of porcine CD47 gene, is generated. One or more selectable marker are included in the plasmid or the vector. A plasmid with Cas9 and one or more plasmids with gRNAs are generated. In some embodiments, the one or more gRNAs target sites upstream the desired substitution region. In other embodiments, the one or more gRNAs target sites downstream the desired substitution region. In yet other embodiments, the one or more gRNAs target sites upstream and downstream of the desired 28dminister28n region. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a plasmid or a vector is generated that contains a human CD47 sequence, flanked by two homology arms, one of which is upstream of the porcine CD47 gene sequence and the other is downstream of the porcine CD47 gene sequence. In some embodiments, one or more selectable marker are included in the plasmid or the vector. A plasmid with Cas9 and at least two plasmids with gRNAs are generated. Two ssODNs are generated. One ssODN comprises (1) the plasmid or vector region that is upstream of the human CD47 sequence; and (2) the beginning of the host CD47 gene. The other ssODN comprises (1) the end of the host CD47 gene; and (2) the plasmid or vector region that is downstream of the human CD47 sequence. In some embodiments, the two or more gRNAs target sites upstream and downstream of the desired 29dminister29n region. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a plasmid or a vector containing a human CD47 sequence flanked by two homology arms, one of which is upstream the porcine CD47 gene sequence and the other of which is the downstream sequence of porcine CD47 gene, is generated. One or more selectable marker are included in the plasmid or the vector. One or more plasmids encoding a ZFN, a ZFN dimer, a ZFNickase are generated. In some embodiments, the one or more ZFNs or ZFNickases target a site upstream the desired substitution region. In other embodiments, the one or more ZFNs or ZFNickases target a site downstream the desired substitution region. In yet other embodiments, the one or more ZFNs or ZFNickases target sites upstream and downstream of the desired substitution region. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a plasmid or a vector containing a human CD47 sequence flanked by two homology arms, one of which is upstream the porcine CD47 gene sequence and the other of which is the downstream sequence of porcine CD47 gene, is generated. One or more selectable marker are included in the plasmid or the vector. One or more plasmids encoding TALENs are generated. In some embodiments, the one or more TALENs target a site upstream the desired substitution region. In other embodiments, the one or more TALENs target a site downstream the desired substitution region. In yet other embodiments, the one or more TALENs target sites upstream and downstream the desired substitution region. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a plasmid with Cas9 and two or more plasmids with gRNAs are generated. In some embodiments, the two or more gRNAs target sites upstream and downstream of the porcine CD47 sequence so that the Cas9 endonucleases cut at a site that is upstream of the start of the porcine CD47 sequence by certain base pairs, and Cas9 endonucleases cut at a site that is downstream of the end of the porcine CD47 sequence by certain base pairs. In some embodiments, the number of base pairs is below 50 bp. In other embodiments, the number of base pairs is 50-100 bp. In yet other embodiments, the number of base pairs is 100-150 bp. In yet other embodiments, the number of base pairs is 150-200 bp. In yet other embodiments, the number of base pairs is above 200 bp. A plasmid or a vector containing a human CD47 sequence flanked by the above-described base pairs plus the two Cas 9 cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, two or more plasmids encoding ZFNs or ZFNickases are generated. In some embodiments, ZFNs or ZFNickases cut at a site that is upstream of the start of the porcine CD47 sequence, and ZFNs or ZFNickases cut at a site that is downstream of the end of the porcine CD47 sequence. In some embodiments, the number of base pairs upstream the start site is below 50 bp. In some embodiments, the number of base pairs downstream the stop site is below 50 bp. In some embodiments, the number of base pairs upstream the start site is 50-200 bp. In some embodiments, the number of base pairs downstream the stop site is 50-200 bp. In some embodiments, the number of base pairs upstream the start site is 50-100 bp. In some embodiments, the number of base pairs downstream the stop site is 50-100 bp. In some embodiments, the number of base pairs upstream the start site is 100-150 bp. In some embodiments, the number of base pairs downstream the stop site is 100-150 bp. In some embodiments, the number of base pairs upstream the start site is 150-200 bp. In some embodiments, the number of base pairs downstream the stop site is 150-200 bp. In some embodiments, the number of base pairs upstream the start site more than 200 bp. In some embodiments, the number of base pairs downstream the stop site is more than 200 bp.

A plasmid or a vector containing a human CD47 sequence flanked by the above-described base pairs plus the two ZFNs or ZFNickases cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, two or more plasmids encoding TALENs are generated. In some embodiments, TALENs cut at a site that is upstream of the start of the porcine CD47 sequence, and downstream of the end of the porcine CD47 sequence by certain base pairs. In some embodiments, the TALENs cut at a site that is less than 50 bp upstream from the start site. In some embodiments, the TALENs cut at a site that is less than 50 bp downstream from the stop site. In some embodiments, the TALENs cut at a site 50-100 bp upstream from the start site. In some embodiments, the TALENs cut at a site 50-100 bp downstream from the stop site. In some embodiments, the TALENs cut at a site 100-150 bp upstream from the start site. In some embodiments, the TALENs cut at a site 100-150 bp downstream from the stop site. In some embodiments, the TALENs cut at a site 150-200 bp upstream from the start site. In some embodiments, the TALENs cut at a site 150-200 bp downstream from the stop site. In some embodiments, the TALENs cut at a site greater than 200 bp upstream from the start site. In some embodiments, the TALENs cut at a site greater than 200 bp downstream from the stop site.

A plasmid or a vector containing a human CD47 sequence flanked by the above-described base pairs plus the two TALENs cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a pair of heterospecific recombinase recognition sites is inserted to flank the porcine CD47 sequence or portion thereof. A plasmid or a vector containing a human CD47 sequence flanked by the identical pair of recombinase recognition sites. One or more selectable marker are included in the plasmid or the vector. A plasmid encoding the corresponding recombinase is generated. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine CD47 sequence or portion thereof. A plasmid or a vector containing a human CD47 sequence flanked by the identical pair of recombinase recognition sites. One or more selectable marker are included in the plasmid or the vector. A plasmid encoding the corresponding recombinase is generated. A plasmid with Cas9 and two or more plasmids with gRNAs are generated. In some embodiments, the two or more gRNAs target the upstream and downstream of the porcine CD47 sequence so that the porcine CD47 sequence is deleted leaving the inserted human CD47 sequence. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine CD47 sequence or portion thereof. A plasmid or a vector containing a human CD47 sequence flanked by the identical pair of recombinase recognition sites. One or more selectable marker are included in the plasmid or the vector. A plasmid encoding the corresponding recombinase is generated. Plasmid encoding pairs of ZENs or ZFNickases are generated. In some embodiments, the ZENs or ZFNickases target the upstream and downstream of the porcine CD47 sequence so that the porcine CD47 sequence is deleted leaving the inserted human CD47 sequence. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine CD47 sequence or portion thereof. A plasmid or a vector containing a human CD47 sequence flanked by the identical pair of recombinase recognition sites. One or more selectable marker are included in the plasmid or the vector. A plasmid encoding the corresponding recombinase is generated. Plasmid encoding a pair of TALENs are generated. In some embodiments, the TALENs target sites upstream and downstream of the porcine CD47 sequence so that the porcine CD47 sequence is deleted, leaving the inserted human CD47 sequence. The above-described plasmids or vectors are transfected into cells, including but not limiting to, cultured porcine fetal fibroblasts. With selection steps that match the selectable marker(s), cells with porcine CD47 successfully substituted by the human CD47 sequence are isolated, expanded, and verified using techniques well-known in the art. SCNT or other equivalent technologies can be used to generate recombinant miniature swine.

In specific embodiments, a plasmid or a vector is generated containing a human SIRPA sequence flanked by two homology arms, one of which is upstream the porcine SIRPA gene sequence and the other of which is downstream the porcine SIRPA gene sequence. In some embodiments, a plasmid or a vector containing a portion of a human SIRPA sequence flanked by two homology arms, one of which is upstream the target portion of the porcine SIRPA gene sequence and the other of which is the downstream the target portion of porcine SIRPA gene, is generated. One or more selectable marker are included in the plasmid or the vector. The above-described plasmid or vector is transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a plasmid or a vector is generated containing a human SIRPA sequence flanked by two homology arms, one of which is upstream the porcine SIRPA gene sequence and the other of which is downstream the porcine SIRPA gene sequence. One or more selectable marker are included in the plasmid or the vector. A plasmid with Cas9 and one or more plasmids with gRNAs are generated. In some embodiments, the one or more gRNAs target sites upstream the desired substitution region. In other embodiments, the one or more gRNAs target sites downstream the desired substitution region. In yet other embodiments, the one or more gRNAs target sites upstream and downstream of the desired 34dminister34n region. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a plasmid or a vector is generated containing a human SIRPA sequence, flanked by two homology arms, one of which is upstream the porcine SIRPA gene sequence and the other of which is downstream the porcine SIRPA gene sequence. One or more selectable marker are included in the plasmid or the vector. A plasmid with Cas9 and at least two plasmids with gRNAs are generated. Two ssODNs are generated. One ssODN comprises (1) the plasmid or vector region that is upstream of the human SIRPA sequence; and (2) the beginning of the host SIRPA gene. The other ssODN comprises (1) the end of the host SIRPA gene; and (2) the plasmid or vector region that is downstream of the human SIRPA sequence. In some embodiments, the two or more gRNAs target sites upstream and downstream of the desired 35dminister35n region. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a plasmid or a vector containing a human SIRPA sequence flanked by two homology arms, one of which is upstream the porcine SIRPA gene sequence and the other of which is downstream the porcine SIRPA gene sequence. One or more selectable markers are included in the plasmid or the vector. One or more plasmids encoding a ZFN, a ZFN dimer, a ZFNickase are generated. In some embodiments, the one or more ZFNs or ZFNickases target a site upstream of the desired substitution region. In other embodiments, the one or more ZFNs or ZFNickases target sites downstream the desired substitution region. In yet other embodiments, the one or more ZFNs or ZFNickases target sites upstream and downstream of the desired 35dminister35n region. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a plasmid or a vector containing a human SIRPA sequence flanked by two homology arms, one of which is upstream the porcine SIRPA gene sequence and the other of which is downstream the porcine SIRPA gene sequence. One or more selectable marker are included in the plasmid or the vector. One or more plasmids encoding TALENs are generated. In some embodiments, the one or more TALENs target sites upstream the desired substitution region. In other embodiments, the one or more TALENs target sites downstream the desired substitution region. In yet other embodiments, the one or more TALENs target sites upstream and downstream of the desired 35dminister35n region. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a plasmid with Cas9 and two or more plasmids with gRNAs are generated. In some embodiments, the two or more gRNAs target the upstream and downstream of the porcine SIRPA sequence so that the Cas9 endonuclease cuts at a site that is upstream of the start of the porcine SIRPA sequence, and Cas9 endonuclease cuts at a site that is downstream of the end of the porcine SIRPA sequence. In some embodiments, the Cas9 endonuclease cuts at a site that is upstream of the start of the porcine SIRPA sequence, and downstream of the end of the porcine SIRPA sequence. In some embodiments, the Cas9 endonuclease cuts at a site that is less than 50 bp upstream from the start site. In some embodiments, the Cas9 endonuclease cuts at a site that is less than 50 bp downstream from the stop site. In some embodiments, the Cas9 endonuclease cuts at a site 50-100 bp upstream from the start site. In some embodiments, the Cas9 endonuclease cuts at a site 50-100 bp downstream from the stop site. In some embodiments, the Cas9 endonuclease cuts at a site 100-150 bp upstream from the start site. In some embodiments, the Cas9 endonuclease cuts at a site 100-150 bp downstream from the stop site. In some embodiments, the Cas9 endonuclease cuts at a site 150-200 bp upstream from the start site. In some embodiments, the Cas9 endonuclease cuts at a site 150-200 bp downstream from the stop site. In some embodiments, the Cas9 endonuclease cuts at a site greater than 200 bp upstream from the start site. In some embodiments, the Cas9 endonuclease cuts at a site greater than 200 bp downstream from the stop site.

A plasmid or a vector containing a human SIRPA sequence flanked by the above-described base pairs plus the two Cas 9 cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, two or more plasmids encoding ZFNs or ZFNickases are generated. In some embodiments, ZFNs or ZFNickases cut at a site that is upstream of the start of the porcine SIRPA sequence, and ZFNs or ZFNickases cut at a site that is downstream of the end of the porcine SIRPA sequence. In some embodiments, ZFNs or ZFNickases cut at a site that is less than 50 bp upstream from the start site. In some embodiments, ZFNs or ZFNickases cut at a site that is less than 50 bp downstream from the stop site. In some embodiments, ZFNs or ZFNickases cut at a site 50-100 bp upstream from the start site. In some embodiments, ZFNs or ZFNickases cut at a site 50-100 bp downstream from the stop site. In some embodiments, ZFNs or ZFNickases cut at a site 100-150 bp upstream from the start site. In some embodiments, ZFNs or ZFNickases cut at a site 100-150 bp downstream from the stop site. In some embodiments, ZFNs or ZFNickases cut at a site 150-200 bp upstream from the start site. In some embodiments, ZFNs or ZFnickases cut at a site 150-200 bp downstream from the stop site. In some embodiments, ZFNs or ZFNickases cut at a site greater than 200 bp upstream from the start site. In some embodiments, ZFNs or ZFNickases cut at a site greater than 200 bp downstream from the stop site.

A plasmid or a vector containing a human SIRPA sequence flanked by the above-described base pairs plus the two ZENs or ZFNickases cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, two or more plasmids encoding TALENs are generated. In some embodiments, TALENs cut at a site that is upstream of the start of the porcine SIRPA sequence, and TALENs cut at a site that is downstream of the end of the porcine SIRPA sequence. In some embodiments, the Cas9 endonuclease cuts at a site that is less than 50 bp upstream from the start site. In some embodiments, TALENs cut at a site that is less than 50 bp downstream from the stop site. In some embodiments, TALENs cut at a site 50-100 bp upstream from the start site. In some embodiments, TALENs cut at a site 50-100 bp downstream from the stop site. In some embodiments, TALENs cut at a site 100-150 bp upstream from the start site. In some embodiments, TALENs cut at a site 100-150 bp downstream from the stop site. In some embodiments, TALENs cut at a site 150-200 bp upstream from the start site. In some embodiments, TALENs cut at a site 150-200 bp downstream from the stop site. In some embodiments, TALENs cut at a site greater than 200 bp upstream from the start site. In some embodiments, TALENs cut at a site greater than 200 bp downstream from the stop site.

A plasmid or a vector containing a human SIRPA sequence flanked by the above-described base pairs plus the two TALENs cutting sites. One or more selectable marker are included in the plasmid or the vector. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a pair of heterospecific recombinase recognition sites is inserted to flank the porcine SIRPA sequence or portion thereof. A plasmid or a vector containing a human SIRPA sequence flanked by the identical pair of recombinase recognition sites. One or more selectable marker are included in the plasmid or the vector. A plasmid encoding the corresponding recombinase is generated. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine SIRPA sequence or portion thereof. A plasmid or a vector containing a human SIRPA sequence flanked by the identical pair of recombinase recognition sites. One or more selectable marker are included in the plasmid or the vector. A plasmid encoding the corresponding recombinase is generated. A plasmid with Cas9 and two or more plasmids with gRNAs are generated. In some embodiments, the two or more gRNAs target the upstream and downstream of the porcine SIRPA sequence so that the porcine SIRPA sequence is deleted leaving the inserted human SIRPA sequence. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine SIRPA sequence or portion thereof. A plasmid or a vector containing a human SIRPA sequence flanked by the identical pair of recombinase recognition sites. One or more selectable marker are included in the plasmid or the vector. A plasmid encoding the corresponding recombinase is generated. Plasmid encoding pairs of ZENs or ZFNickases are generated. In some embodiments, the ZENs or ZFNickases target the upstream and downstream of the porcine SIRPA sequence so that the porcine SIRPA sequence is deleted leaving the inserted human SIRPA sequence. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In other specific embodiments, a loading pad flanked by a pair of heterospecific recombinase recognition sites is first inserted near the porcine SIRPA sequence or portion thereof. A plasmid or a vector containing a human SIRPA sequence flanked by the identical pair of recombinase recognition sites. One or more selectable marker are included in the plasmid or the vector. A plasmid encoding the corresponding recombinase is generated. Plasmid encoding a pair of TALENs are generated. In some embodiments, the TALENs target the upstream and downstream of the porcine SIRPA sequence so that the porcine SIRPA sequence is deleted leaving the inserted human SIRPA sequence. The above-described plasmids or vectors are transfected into cells, including but not limiting to, cultured porcine fetal fibroblasts. With selection steps that match the selectable marker(s), cells with porcine SIRPA successfully substituted by the human SIRPA sequence are isolated, expanded, and verified using techniques well-known in the art. SCNT or other equivalent technologies can be used to generate recombinant miniature swine.

In an another aspect, provided herein is a method of generating a recombinant miniature swine, wherein the endogenous porcine CD47 or portion thereof is genetically modified in a way so that its protein product can functionally bind to and activate both the endogenous porcine SIRPA and human SIRPA to elicit the negative regulation of phagocytosis by macrophages.

### 7.1.3 Methods of Testing Expression of Transgenes in the Recombinant Miniature Swine

Various methods for testing and validating expression of the transgenes can be used, and are known in the art. In certain embodiments, levels of human or humanized CD47 and SIRPA expression can be determined at the RNA (e.g., mRNA) level, such as by the methods described in Section 7.1.3.1. In certain embodiments, levels of human or humanized CD47 and SIRPA expression can be determined at the protein level such as by the methods described in Section 7.1.3.2.

In certain embodiments, the methods provided herein include methods of detecting and measuring differential gene expression in any cells, tissues, or organs of the donor miniature swine. In certain embodiments, the methods provided herein include methods of detecting and measuring differential mRNA levels of human or humanized CD47 and SIRPA in any cells, tissues, or organs of the donor miniature swine. In other embodiments, the methods provided herein include methods of detecting and measuring differential protein levels of human or humanized CD47 and SIRPA in any cells, tissues, or organs of the donor miniature swine.

Tissue-specific expression may be determined by physically isolating the tissue of interest before measuring the protein or mRNA levels of human or humanized CD47 and SIRPA (e.g., biopsy of different tissues or organs, or flow cytometry of certain cell types) and applying methods to measure the protein or mRNA levels of human or humanized CD47 and SIRPA such as the ones below in vitro. Alternatively, imagining techniques such as fluorescent microscopy may be used to visualize and measure the protein expression of human or humanized CD47 and SIRPA in specific tissues if visualization labels are engineered to be expressed concurrently with human or humanized CD47 and SIRPA genes. Single cell qPCR may be used to measure human or humanized CD47 and SIRPA gene expression in specific tissues.

### 7.1.3.1 Methods of Detecting mRNA Levels in the Recombinant Miniature Swine

In certain embodiments, mRNA of human or humanized CD47 and SIRPA from the recombinant miniature swine described in Sections 7.1.1 and 7.1.2 is detected by a technique described herein. In some embodiments, mRNA of porcine CD47 and SIRPA from the recombinant miniature swine described in Section 7.1.1 and 7.1.2 is not detectable using a technique described herein.

Several methods of detecting or quantifying mRNA levels are known in the art. Exemplary methods include, but are not limited to, northern blots, ribonuclease protection assays, PCR-based methods (e.g., quantitative PCR), RNA sequencing, Fluidigm® analysis, and the like. The mRNA sequence of a human or humanized CD47 and SIRPA can be used to prepare a probe that is at least partially complementary to the specific fragments of human mRNA sequence, but not complementary to the porcine counterparts. Similarly, the mRNA sequence of porcine CD47 and SIRPA can be used to prepare a probe that is at least partially complementary to the specific fragments of porcine mRNA sequence, but not complementary to the human counterparts. The probes can then be used to detect the presence of mRNA of human or humanized and porcine CD47 and SIRPA in a sample, using any suitable assay, such as PCR-based methods, northern blotting, a dipstick assay, TaqMan™ assays and the like.

In other embodiments, a nucleic acid assay for testing for human or humanized CD47 and SIRPA expression in a biological sample can be prepared. An assay typically contains a solid support and at least one nucleic acid contacting the support. The nucleic acid corresponds to at least a portion of the mRNA that is unique to human CD47 and SIRPA, but absent in the porcine counterparts. Similarly, the nucleic acid corresponds to at least a portion of the mRNA that is unique to porcine CD47 and SIRPA, but absent in the human counterparts. The assay can also have a means for detecting the altered expression of the mRNA in the sample. The assay method can be varied depending on the type of mRNA information desired. Exemplary methods include but are not limited to Northern blots and PCR-based methods (e.g., qRT-PCR). Methods such as qRT-PCR can also accurately quantitate the amount of the mRNA in a sample.

A typical mRNA assay method can contain the steps of: (1) obtaining surface-bound subject probes; (2) hybridizing a population of mRNAs to the surface-bound probes under conditions sufficient to provide for specific binding; (3) post-hybridization washing to remove nucleic acids not specifically bound to the surface-bound probes; and (4) detecting the hybridized mRNAs. The reagents used in each of these steps and their conditions for use may vary depending on the particular application.

Other methods, such as PCR-based methods, can also be used to detect the expression of human or humanized CD47 and SIRPA. Examples of PCR methods can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

In some embodiments, quantitative Reverse Transcription-PCR (qRT-PCR) can be used for both the detection and quantification of RNA targets (Bustin et al., Clin. Sci. 2005, 109:365-379). In some embodiments, qRT-PCR-based assays can be useful to measure mRNA levels during cell-based assays. Examples of qRT-PCR-based methods can be found, for example, in U.S. Pat. No. 7,101,663, which is incorporated by reference herein in its entirety.

In contrast to regular reverse transcriptase-PCR and analysis by agarose gels, qRT-PCR gives quantitative results. An additional advantage of qRT-PCR is the relative ease and convenience of use. Instruments for qRT-PCR, such as the Applied Biosystems 7500, are available commercially, so are the reagents, such as TaqMan® Sequence Detection Chemistry. For example, TaqMan® Gene Expression Assays can be used, following the manufacturer's instructions. These kits are pre-formulated gene expression assays for rapid, reliable detection and quantification of human, mouse, and rat mRNA transcripts. An exemplary qRT-PCR program, for example, is 50° C. for 2 minutes, 95° C. for 10 minutes, 40 cycles of 95° C. for 15 seconds, then 60° C. for 1 minute.

### 7.1.3.2 Methods of Detecting Polypeptide or Protein Levels in the Recombinant Miniature Swine

In one aspect, provided herein is a method to detect human or humanized CD47 and SIRPα polypeptide or protein generated in the recombinant miniature swine, such as the recombinant miniature swine described in Sections 7.1.1 and 7.1.2. In some embodiments provided herein, porcine CD47 and SIRPα polypeptide or protein is not detectable in the recombinant miniature swine described in 7.1.1 and 7.1.2 described herein.

Various protein detection and quantification methods can be used to measure the level of human or humanized CD47 and SIRPα. Any suitable protein quantification method can be used. In some embodiments, antibody-based methods are used. Exemplary methods that can be used include, but are not limited to, immunoblotting (Western blot), ELISA, immunohistochemistry, immunofluorescence, flow cytometry, cytometry bead array, mass spectroscopy, and the like. Several types of ELISA are commonly used, including direct ELISA, indirect ELISA, and sandwich ELISA.

### 7.1.4 Other Genetic Modifications

Recombinant miniature swine provided herein may be further modified in addition to CD47 and SIRPA. Such additional modifications include, for example, knockout of α-1,3-galactosyltransferase and modifications of the cytokine receptors. In some embodiment, a miniature swine provided herein does not express α-1,3-galactosyltransferase. In some embodiments, a miniature swine provided herein additionally expresses human CD55, human CD46, human CD59, IL-3R, or some combination thereof. See, e.g., Nomura et al. (2020), Xenotransplantation. 2020; 27:e12549, U.S. Pat. Nos. 9,883,939 and 9,980,471 B2.

Referring to the transplantation methods in Section 7.3, such additional genetic modifications can be used in connection with the miniature swine that is the donor for an organ transplant, and such additional modifications can also be used in connection with the miniature swine that is the donor for hematopoietic stem cells (e.g., for a bone marrow transplant).

### 7.2 Cells, Tissues, Organs Derived from the Recombinant Miniature Swine

Cells, tissues, organs or body fluids of the transgenic donor miniature swine, as described in Section 7.1, may be used in methods of transplantation (e.g., xenotransplantation).

In some embodiments, cells of transgenic donor miniature swine, as described in Section 7.1, may be used in methods of transplantation (e.g., xenotransplantation). In some specific embodiments, erythrocytes from transgenic donor miniature swine are used. In some specific embodiments, granulocytes from transgenic donor miniature swine are used. In some specific embodiments, a granulocytes from transgenic donor miniature swine are used. In some specific embodiments, platelets from transgenic donor miniature swine are used. In some specific embodiments, neurons from transgenic donor miniature swine are used. In some specific embodiments, glial cells from transgenic donor miniature swine are used. In some specific embodiments, muscle cells from transgenic donor miniature swine are used. In some specific embodiments, chondrocytes from transgenic donor miniature swine are used. In some specific embodiments, bone cells from transgenic donor miniature swine are used. In some specific embodiments, skin cells from transgenic donor miniature swine are used. In some specific embodiments, endothelial cells from transgenic donor miniature swine are used. In some specific embodiments, epithelial cells from transgenic donor miniature swine are used. In some specific embodiments, adipocytes from transgenic donor miniature swine are used. In some specific embodiments, spermatozoa from transgenic donor miniature swine are used. In some specific embodiments, ova from transgenic donor miniature swine are used.

In some embodiments, tissues of transgenic donor miniature swine, as described in Section 7.1, may be used in methods of transplantation (e.g., xenotransplantation). In some specific embodiments, connective tissues from transgenic donor miniature swine are used. In some specific embodiments, epithelial tissues from transgenic donor miniature swine are used. In some specific embodiments, muscle tissues from transgenic donor miniature swine are used. In some specific embodiments, nervous tissues from transgenic donor miniature swine are used.

In some embodiments, organs of transgenic donor miniature swine, as described in Section 7.1, may be used in methods of transplantation (e.g., xenotransplantation). In some specific embodiments, skeletons from transgenic donor miniature swine are used. In some specific embodiments, joints from transgenic donor miniature swine are used. In some specific embodiments, ligaments from transgenic donor miniature swine are used. In some specific embodiments, tendons from transgenic donor miniature swine are used. In some specific embodiments, salivary glands from transgenic donor miniature swine are used. In some specific embodiments, esophaguses from transgenic donor miniature swine are used. In some specific embodiments, tracheas from transgenic donor miniature swine are used. In some specific embodiments, stomachs from transgenic donor miniature swine are used. In some specific embodiments, small intestines from transgenic donor miniature swine are used. In some specific embodiments, large intestines from transgenic donor miniature swine are used. In some specific embodiments, livers from transgenic donor miniature swine are used. In some specific embodiments, gallbladders from transgenic donor miniature swine are used. In some specific embodiments, mesenteries from transgenic donor miniature swine are used. In some specific embodiments, pancreases from transgenic donor miniature swine are used. In some specific embodiments, lungs from transgenic donor miniature swine are used. In some specific embodiments, hearts from transgenic donor miniature swine are used. In some specific embodiments, islets from transgenic donor miniature swine are used. In some specific embodiments, kidneys from transgenic donor miniature swine are used. In some specific embodiments, bladders from transgenic donor miniature swine are used. In some specific embodiments, urethras from transgenic donor miniature swine are used. In some specific embodiments, uteruses from transgenic donor miniature swine are used. In some specific embodiments, pituitary glands from transgenic donor miniature swine are used. In some specific embodiments, pineal glands from transgenic donor miniature swine are used. In some specific embodiments, thyroid glands from transgenic donor miniature swine are used. In some specific embodiments, parathyroid glands from transgenic donor miniature swine are used. In some specific embodiments, skin from transgenic donor miniature swine are used. In some specific embodiments, adrenal glands from transgenic donor miniature swine are used. In some specific embodiments, arteries from transgenic donor miniature swine are used. In some specific embodiments, veins from transgenic donor miniature swine are used. In some specific embodiments, capillaries from transgenic donor miniature swine are used. In some specific embodiments, lymphatic vessels from transgenic donor miniature swine are used. In some specific embodiments, lymph nodes from transgenic donor miniature swine are used. In some specific embodiments, bone marrows from transgenic donor miniature swine are used. In some specific embodiments, thymuses from transgenic donor miniature swine are used. In some specific embodiments, spleens from transgenic donor miniature swine are used. In some specific embodiments, corneas from transgenic donor miniature swine are used. In some specific embodiments, retinas from transgenic donor miniature swine are used. In some specific embodiments, irises from transgenic donor miniature swine are used.

### 7.3 Methods of Transplantation

Provided herein are methods of transplanting a graft from a first donor such as the recombinant miniature swine described in Section 7.1 with or without bone marrow from a second donor such as the recombinant miniature swine described in Section 7.1. Specifically, methods of obtaining cells, tissues, and organs of a transgenic donor miniature swine are described in Section 7.3.1. The preparation of the transplantation, the process of transplantation, and procedures after the transplantation are described in Section 7.3.2. The recipient patient groups are described in Section 7.3.3. Tolerance of xenografts can be improved by the methods described in Section 7.3.4. The outcome of transplantation can be measured as described in Section 7.3.7.

### 7.3.1 Methods of Obtaining a Graft

In some aspect, the method provided herein comprises obtaining a graft from a recombinant miniature swine as described in Section 7.1.

It is well known in the art regarding the surgery procedures of obtaining a graft from a miniature swine. For example, Tena et al. (2017), Transplantation 101:316-21; and Nomura et al. (2020), Xenotransplantation 2020; 27:e12549. The contents of the above are incorporated herein by reference in its entirety.

In some embodiments, during the transition period after the graft is removed from the donor animal but before being transplanted in the recipient, the graft is stored in certain container to keep the viability of the graft. In some specific embodiments, the container is capable of perfusion of the graft at warm and/or cold temperatures to extend the useful life of the graft. In some specific embodiments, the graft is stored in the container with proper fluid samples. In some specific embodiments, the graft is stored in the container with ample supply of oxygen. U.S. Pat. No. 6,673,594, which is hereby incorporated by reference in its entirety.

### 7.3.2 Methods of Transplanting a Graft

In some aspect, the method of transplanting a graft from a recombinant miniature swine to a recipient provided herein comprises (a) obtaining the graft from the recombinant miniature swine as described in Section 7.1; and (b) transplanting the graft to the primate. In some embodiments, the recipient is a primate. In some specific embodiments, the recipient is a human. In some embodiments, the graft comprises cells, a tissue, or an organ, wherein the organ can be a heart, a kidney, an islet, a liver, a pancreas, a lung, an intestine, skin, a small bowel, a trachea, a cornea, or combination thereof.

In some aspect, the method of transplantation provided herein comprises (a) obtaining bone marrow from a recombinant miniature swine as described in Section 7.1; (b) transplanting the bone marrow to a recipient; (c) obtaining the graft from another recombinant miniature swine as described in Section 7.1 to the same recipient; and (d) transplanting the graft to the same recipient.

In some embodiments, the hematopoietic stem cells and the donor cells, tissues, and organs are taken from the same donor animal. In other embodiments, the hematopoietic stem cells and the donor cells, tissues, and organs are taken from two different, but genetically matched donor animals. “Genetically matched” as used herein may refer to homology between genes, for example, MHC genes. In some embodiments, the genetically matched donor animals are perfectly matched for MHC. In some embodiments, the hematopoietic stem cells and the donor cells, tissues, and organs are taken from two different animals from the same, highly inbred herd.

In certain embodiments, the step of transplanting donor cells, tissues, and organs from a miniature swine is carried out at least 7 days after the step of transplanting hematopoietic stem cells (e.g., bone marrow) from another miniature swine. In certain embodiments, the step of transplanting donor cells, tissues, and organs from a miniature swine is carried out at least 14 days after the step of transplanting hematopoietic stem cells (e.g., bone marrow) from another miniature swine. In certain embodiments, the step of transplanting donor cells, tissues, and organs from a miniature swine is carried out at least 21 days after the step of transplanting hematopoietic stem cells (e.g., bone marrow) from another miniature swine. In certain embodiments, the step of transplanting donor cells, tissues, and organs from a miniature swine is carried out at least 28 days after the step of transplanting hematopoietic stem cells (e.g., bone marrow) from another miniature swine. In certain embodiments, the step of transplanting donor cells, tissues, and organs from a miniature swine is carried out at least 35 days after the step of transplanting hematopoietic stem cells (e.g., bone marrow) from another miniature swine. In certain embodiments, the step of transplanting donor cells, tissues, and organs from a miniature swine is carried out at least 42 days after the step of transplanting hematopoietic stem cells (e.g., bone marrow) from another miniature swine. In certain embodiments, the step of transplanting donor cells, tissues, and organs from a miniature swine is carried out at least 49 days after the step of transplanting hematopoietic stem cells (e.g., bone marrow) from another miniature swine. In certain embodiments, the step of transplanting donor cells, tissues, and organs from a miniature swine is carried out at least 56 days after the step of transplanting hematopoietic stem cells (e.g., bone marrow) from another miniature swine. The present disclosure includes the methods and techniques described in Watanabe et al., Xenotransplantation, 2020, 27:e12552 and Nomura et al., Xenotransplantation, 2020, 27:e12549 for transgenic expression of human CD47 in donor cells.

The hematopoietic stem cells can be any type of cell. In certain embodiments, the cell is a hematopoietic stem cell, lymphocyte, or a myeloid cell. In some embodiments, a mixed population of hematopoietic cells is transplanted from the first donor animal (e.g., miniature swine) into the recipient. In certain embodiments, the porcine hematopoietic stem cells are obtained from bone marrow, peripheral blood, umbilical cord blood, fetal liver or embryonic stem cells. The hematopoietic stem cells may be transplanted by any suitable method known in the art, for example by a method described in Section 7.3.4.3 below. In some embodiments, the hematopoietic stem cells are transplanted to the recipient by intra bone-bone marrow transplantation, e.g. as described in Watanabe et al. (2019), Xenotransplantation. 2019; 00:e12552.

### 7.3.3 The Recipients of Transplantation

In a preferred embodiment, a patient treated in accordance with the methods described herein (e.g., the recipient of one or more donor grafts) is a human patient. As used herein, the terms “subject” and “patient” are used interchangeably and include any human or non-human mammal. Non-limiting examples include members of the human, equine, porcine, bovine, Rattus, murine, canine and feline species. In some embodiments, the subject is a non-human primate. In some embodiments, the subject is human. In specific embodiments, the subject is a human adult. In some embodiments, the subject is a human child. In specific embodiments, the subject is human and receives one or more donor grafts from a porcine donor. In other specific embodiments, the subject is a non-human primate (e.g., a baboon, a cynomolgus monkey or a rhesus macaque) and receives one or more grafts from a porcine donor.

In some embodiments, a patient treated in accordance with the methods described herein is in need of a kidney transplant. A patient may be in need of a kidney transplant due to renal failure or the rejection of a donor kidney. Renal failure can have a number of causes, including but not limited to high blood pressure (hypertension), physical injury, diabetes, kidney disease (polycystic kidney disease, glomerular disease) and autoimmune disorders such as lupus. Renal failure may be acute or chronic. Kidney failure can also be diagnosed by laboratory tests such as glomerular filtration rate, blood urea nitrogen, and serum creatinine, by imaging test (ultrasound, computer tomography) or a kidney biopsy. In some embodiments, a patient treated in accordance with a method described herein has Stage 1, 2, 3, 4, or 5 kidney disease.

In some embodiments, a patient treated in accordance with the methods described herein is in need of a heart transplant. A patient may be in need of a heart transplant due to heart failure or coronary artery disease. Heart failure or coronary artery disease can have a number of causes, including but not limited to dilated cardiomyopathy, restrictive myopathy, hypertrophic cardiomyopathy, valvular heart disease, congenital heart disease, ventricular arrhythmias. End-stage heart failure or severe coronary artery disease may be acute or chronic. Heart failure and coronary artery disease can be diagnosed by laboratory tests such as blood tests, chest X-ray, electrocardiogram (ECG), echocardiogram, stress test, cardiac computerized tomography (CT) scan, magnetic resonance imaging (MRI), coronary angiogram, myocardial biopsy, cardiac catheterization, and angiogram.

In some embodiments, a patient treated in accordance with the methods described herein is in need of an islet transplant. A patient may be in need of an islet transplant due to a lack of capacity of producing insulin, which can have a number of causes, including but not limited to type 1 diabetes. The patients can be diagnosed by laboratory tests such as glycated hemoglobin (A1C) test, random blood sugar test, and fasting blood sugar test.

In some embodiments, a patient treated in accordance with the methods described herein is in need of a liver transplant. A patient may be in need of a liver transplant due to liver failure or liver cancer. Liver failure or liver cancer can have a number of causes, including but not limited to cirrhosis, hepatitis B infection, hepatitis C infection, alcoholic liver disease, nonalcoholic fatty liver disease, genetic diseases affecting the liver, such as but not limited to hemochromatosis and Wilson's disease, primary biliary cirrhosis, primary sclerosing cholangitis and biliary atresia. Liver failure may be acute or chronic. Liver failure can be diagnosed by laboratory tests such as blood tests, ultrasound, CT scan, MRI, and liver biopsy.

In some embodiments, a patient treated in accordance with the methods described herein is in need of a pancreas transplant. A patient may be in need of a pancreas transplant due to a lack of capacity of producing insulin, which can have a number of causes, including but not limited to type 1 diabetes. The patients can be diagnosed by laboratory tests such as glycated hemoglobin (A1C) test, random blood sugar test, and fasting blood sugar test.

In some embodiments, a patient treated in accordance with the methods described herein is in need of a lung transplant. A patient may be in need of a lung transplant due to lung failure or lung cancer. Lung failure or lung cancer can have a number of causes, including but not limited to chronic obstructive pulmonary disease (COPD), including emphysema, pulmonary embolism, pulmonary fibrosis, pulmonary hypertension, and cystic fibrosis. Lung failure may be acute or chronic. Lung failure can be diagnosed by laboratory tests such as physical exams, tests with pulse oximetry, and arterial blood gas test.

In some embodiments, a patient treated in accordance with the methods described herein is in need of an intestine transplant. A patient may be in need of an intestine transplant due to intestinal failure. Intestinal failure can have a number of causes, including but not limited to short bowel syndrome (SBS), chronic intestinal pseudo-obstruction (CIPO), intra-abdominal non-metastasizing tumors, ischemia, Crohn's Disease, trauma, motility disorder, volvulus, necrotizing enterocolitis, gastroschisis, omphalocele, intestinal atresia, microvillus inclusion disease, intractable diarrhea of infancy, autoimmune enteritis, and intestinal polyposis. Intestinal failure may be acute or chronic. Intestinal failure can be diagnosed by laboratory tests such as abdominal computed tomography (CT) scan, abdominal X-ray, barium enema/lower gastrointestinal series, blood tests, colonoscopy, sigmoidoscopy, gastric emptying study, gastroduodenal manometry, scintigraphic gastric accommodation, upper endoscopy, wireless capsule gastrointestinal monitoring system.

In some embodiments, a patient treated in accordance with the methods described herein is in need of a skin transplant. A patient who may be in need of a skin transplant may due to a number of causes, including but not limited to skin infections, deep burns, large and open wounds, bed sores, ulcers on the skin, or skin cancer.

In some embodiments, a patient treated in accordance with the methods described herein is in need of a trachea transplant. A patient may be in need of a trachea transplant due to damaged airways. Damaged airways can have a number of causes, including but not limited to tuberculosis, mucoepidermoid carcinoma, adenoid cystic carcinoma, bronchomalacia, tracheoesophageal fistula, and tracheostomy.

In some embodiments, a patient treated in accordance with the methods described herein is in need of a cornea transplant. A patient may be in need of a cornea transplant due to severe vision impairments. Severe vision impairments can have a number of causes, including but not limited to infections of eyes, inflammation of eyes, thinning cornea, degenerative vision diseases, such as Fuchs' Dystrophy, keratoconus, corneal perforation, corneal scarring, and bullous keratopathy. Severe vision impairments may be acute or chronic. Severe vision impairments can be diagnosed by laboratory tests such as comprehensive eye exam and corneal topography.

In some embodiments, a patient treated in accordance with the methods described herein is in need of a vascular-tissue transplant. A patient may be in need of a vascular-tissue transplant due to poorly functioning, diseased or missing vessels. The types of donated vascular tissue can be, but not limited to the saphenous veins and femoral vessels from the lower extremities and the aortoiliac artery from the abdomen. The causes to receive a vascular-tissue transplant can be a number, including but not limited to peripheral vascular disease, chronic dialysis treatment, severe clotting, and abdominal aortic aneurysm.

### 7.3.4 Methods of Improving Tolerance of Xenografts

Additional treatments may be used prior to, concurrently with, or subsequent to the methods of transplantation described herein. Additional treatments are generally intended to improve the tolerance of the xenograft in recipients, but other treatments are contemplated. A method of transplantation provided herein can thus include administering one or more additional treatments, e.g., a treatment that inhibits T cells, blocks complement, or otherwise down regulates the recipient immune response to the graft.

In some embodiments, a recipient is thymectomized and/or splenectomized.

In some embodiments, a recipient receives radiation, for example, total body irradiation. In specific embodiments, a recipient receives 5-10 Gy or 10-15 Gy irradiation. In some embodiments, thymic irradiation can be used. In some embodiments, the recipient is administered low dose radiation (e.g., a sub lethal dose of between 100 rads and 400 rads whole body radiation). Local thymic radiation may also be used.

The blood of a subject undergoing transplantation by a method described herein may contain antibodies that target the xenograft. Such antibodies can be eliminated by organ perfusion, and/or transplantation of tolerance-inducing bone marrow. Natural antibodies can be absorbed from the recipient's blood by hemoperfusion of a liver of the donor species. Similarly, antibody-producing cells may be present in the recipient. Such antibody producing cells may be eliminated by, for example, irradiation or drug treatments. In certain embodiment, the graft, cells, tissues, or organs used for transplantation may be genetically modified such that they are not recognized by antibodies present in the host (e.g., the cells are α-1,3-galactosyltransferase deficient) per Section 7.1.4.

In some embodiments, donor stromal tissue is administered. In certain embodiments, the donor stromal tissue is obtained from fetal liver, thymus, and/or fetal spleen, and implanted into the recipient, e.g., in the kidney capsule.

### 7.3.4.1 Immunosuppressive Therapy

In some embodiments, the patient receiving a xenograft in accordance with the methods described herein receives immunosuppressive therapy. The immunosuppressive therapy may be any FDA-approved treatment indicated to reduce transplant rejection and/or ameliorate the outcome of xenotransplantation. Non-limiting examples of immunosuppressive therapy include calcineurin inhibitors (e.g., tacrolimus or cyclosporine), antiproliferative agents (e.g., anti-metabolites such a mycophenolate, 6-mercaptopurine or its prodrug azathioprine), inhibitors of mammalian target of rapamycin (mTOR) (e.g., sirolimus, rapamycin), steroids (e.g., prednisone), cell cycle inhibitors (azathioprine or mycophenolate mofetil), lymphocyte-depleting agents (e.g., anti-thymocyte globulin or antibodies such as alemtuzumab, siplizumab or basiliximab) and co-stimulation blockers (e.g., belatacept). See, e.g., Chung et al (2020)., Ann Transl Med. March; 8(6): 409; van der Mark et al. (2020), Eur Respir Rev; 29: 190132 and Benvenuto et al. (2018), J Thorac Dis 10:3141-3155.

Immunosuppressive therapy may be administered as induction therapy (perioperative, or immediately after surgery) a maintenance dose or for an acute rejection. Induction therapy commonly includes basiliximab, anti-thymocyte globulin or alemtuzumab. Immunosuppressive therapy may also be administered as maintenance therapy, which is often required to continue for the life of the recipient. Maintenance immunosuppressive therapy commonly includes a calcineurin inhibitor (tacrolimus or cyclosporine), an antiproliferative agent (mycophenolate or azathioprine), and corticosteroids. Immunosuppressive therapy for acute rejections commonly includes thymoglobulin or mycophenolate. See, e.g., Chung et al. (2020), Ann Transl Med. March; 8: 409 and Benvenuto et al., (2018) J Thorac Dis 10:3141-3155.

Non-limiting examples of immunosuppressants include, (1) antimetabolites, such as purine synthesis inhibitors (such as inosine monophosphate dehydrogenase (IMPDH) inhibitors, e.g., azathioprine, mycophenolate, and mycophenolate mofetil), pyrimidine synthesis inhibitors (e.g., leflunomide and teriflunomide), and antifolates (e.g., methotrexate); (2) calcineurin inhibitors, such as tacrolimus, cyclosporine A, pimecrolimus, and voclosporin; (3) TNF-alpha inhibitors, such as thalidomide and lenalidomide; (4) IL-1 receptor antagonists, such as anakinra; (5) mammalian target of rapamycin (mTOR) inhibitors, such as rapamycin (sirolimus), deforolimus, everolimus, temsirolimus, zotarolimus, and biolimus A9; (6) corticosteroids, such as prednisone; and (7) antibodies to any one of a number of cellular or serum targets (including anti-lymphocyte globulin and anti-thymocyte globulin).

Non-limiting exemplary cellular targets and their respective inhibitor compounds include, but are not limited to, complement component 5 (e.g., eculizumab); tumor necrosis factors (TNFs) (e.g., infliximab, adalimumab, certolizumab pegol, afelimomab and golimumab); IL-5 (e.g., mepolizumab); IgE (e.g., omalizumab); BAYX (e.g., nerelimomab); interferon (e.g., faralimomab); IL-6 (e.g., elsilimomab); IL-12 and IL-13 (e.g., lebrikizumab and ustekinumab); CD3 (e.g., muromonab-CD3, otelixizumab, teplizumab, visilizumab); CD4 (e.g., clenoliximab, keliximab and zanolimumab); CDI 1a (e.g., efalizumab); CD18 (e.g., erlizumab); CD20 (e.g., afutuzumab, ocrelizumab, pascolizumab); CD23 (e.g., lumiliximab); CD40 (e.g., teneliximab, toralizumab); CD62L/L-selectin (e.g., aselizumab); CD80 (e.g., galiximab); CD147/basigin (e.g., gavilimomab); CD154 (e.g., ruplizumab); BlyS (e.g., belimumab); CTLA-4 (e.g., ipilimumab, tremelimumab); CAT (e.g., bertilimumab, lerdelimumab, metelimumab); integrin (e.g., natalizumab); IL-6 receptor (e.g., tocilizumab); LFA-1 (e.g., odulimomab); and IL-2 receptor/CD25 (e.g., basiliximab, daclizumab, inolimomab).

In some embodiments, the methods of transplantation provided herein comprise steps to induce tolerance in the recipient, e.g., by inducing mixed chimerism. “Mixed chimerism” is commonly understood to describe a state in which the lymphohematopoietic system of the recipient of allogeneic hematopoietic stem cells comprises a mixture of host and donor cells. This state is usually attained through either bone marrow or mobilized peripheral blood stem cell transplantation. Mixed chimerism may be transient or stable. See, e.g., Sachs et al. (2014), Cold Spring Harb Perspect Med 2014; 4:a015529; U.S. Pat. Nos. 6,296,846 and 6,306,651. Mixed chimerism may also be achieved by concurrent transplantation of thymic tissue from the donor animal. See, e.g., International Patent Application Publication No. WO2020/061272.

### 7.3.4.2 Vascularized Thymic Transplant

In some embodiments, a patient treated in accordance with a method described herein receives a vascularized thymic transplant. See, e.g., International Patent Application Publication No. PCT WO2020/061272. Thymic tissue can be prepared for transplantation by implantation under the autologous kidney capsule for revascularization. A vascularized thymic transplant can be, for example, a “thymokidney,” i.e., a kidney prepared by transplanting thymic tissue from a donor under the donor's own kidney capsule. See, e.g., Yamada et. al., Transplantation 68(11):1684-1692 (1999), Yamada et al., J Immunol 164:3079-3086 (2000) and Yamada et al., Transplantation 76(3):530-536 (2003). A vascularized thymic transplant can also be a vascularized thymic lobe transplanted separately from the kidney. See, e.g., LaMattina et al., Transplantation 73(5):826-831 (200) and Kamano et al., Proc Natl Acad Sci USA 101(11):3827-3832 (2004).

### 7.3.4.3 Hematopoietic Stem Cell Transplant

Stem cell engraftment and hematopoiesis across disparate species barriers may be enhanced by providing a hematopoietic stromal environment from the donor species. The stromal matrix supplies species-specific factors that are required for interactions between hematopoietic stem cells and their stromal environment, such as hematopoietic growth factors, adhesion molecules, and their ligands.

As liver is the major site of hematopoiesis in the fetus, fetal liver can also serve as an alternative to bone marrow as a source of hematopoietic stem cells. As an alternative or an adjunct to implantation, fetal liver cells can be administered in fluid suspension. The thymus is the major site of T cell maturation. Each organ includes an organ specific stromal matrix that can support differentiation of the respective undifferentiated stem cells implanted into the host. Thymic stromal tissue can be irradiated prior to transplantation.

Porcine hematopoietic chimeras can lead to donor-specific nonresponsiveness in the mixed lymphocyte reaction, lack of antidonor IgG antibody production, and acceptance of donor grafts. Accordingly, mixed chimerism is capable of inducing tolerance in a highly disparate xenogeneic combination and can have the clinical potential to prevent xenograft rejection. See, e.g., Griesemer et al., Immunol. Rev. 2014; 258(1): 241-258; Sachs et al. (2014), Cold Spring Harb Perspect Med 2014; 4:a015529. Bone marrow cells (BMC), or another source of hematopoietic stem cells, e.g., a fetal liver suspension, of the donor can be injected into the recipient in order to induce mixed chimerism. The hematopoietic stem cells may be taken from any source, for example from the bone marrow or peripheral blood stem cells. See, e.g., Sachs et al. (2014), Cold Spring Harb Perspect Med 2014; 4:a015529. Donor BMC home to appropriate sites of the recipient and grow contiguously with remaining host cells and proliferate, forming a chimeric lymphohematopoietic population. By this process, newly forming B cells (and the antibodies they produce) are exposed to donor antigens, so that the transplant will be recognized as self Tolerance to the donor is also observed at the T cell level in animals in which hematopoietic stem cell, e.g., bone marrow cell, engraftment has been achieved. Transplantation of thymic tissue (e.g., vascularized thymus or a thymokidney) can induce T cell tolerance by generating a T cell repertoire that is not reactive to a xenograft. The use of xenogeneic donors allows the possibility of using bone marrow cells and organs from the same animal, or from genetically matched animals. For bone marrow transplant, the recipient can be administered low dose radiation. In some cases, the recipient can be treated with an agent that depletes complement, such as cobra venom factor (e.g., at day −1).

### 7.3.5 Methods of Evaluating Transplantation

In some embodiments, xenotransplantation using a transplant produced according to the present disclosure results in reduced administration of immunosuppressive therapy to the recipient when compared to current standard of care after the transplantation of the donor cells, tissues, and/or organs. In some embodiments, xenotransplantation using a transplant produced according to the present disclosure results in decreased occurrences of rejection of the donor cells, tissues, and/or organs in a short term when compared to current standard of care. In some embodiments, xenotransplantation using a transplant produced according to the present disclosure results in decreased occurrences of rejection of the donor cells, tissues, and/or organs in a long term when compared to current standard of care. In some embodiments, xenotransplantation using a transplant produced according to the present disclosure results in more prolonged viability of the donor cells, tissues, and/or organ when compared to current standard of care. In some embodiments, xenotransplantation using a transplant produced according to the present disclosure results in better function of the donor cells, tissues, and/or organs in the recipients who receive the donor cells, tissues, and/or organs when compared to current standard of care. In some embodiments, xenotransplantation using a transplant produced according to the present disclosure results in improvements of the corresponding disease that the transplantation aims to intervene with in the recipients who receive the donor cells, tissues, and/or organs, indicated by the corresponding biomarker(s), when compared to current standard of care. In some embodiments, xenotransplantation using a transplant produced according to the present disclosure results in normal functions of other untargeted organ systems in the recipients who receive the donor cells, tissues, and/or organs. In some embodiments, xenotransplantation using a transplant produced according to the present disclosure results in better survival of the recipients who receive the donor cells, tissues, and/or organs when compared to current standard of care. In some embodiments, xenotransplantation using a transplant produced according to the present disclosure results in better quality of life of the recipients who receive the donor cells, tissues, and/or organs when compared to current standard of care.

In specific embodiments, xenotransplantation using a transplant produced according to the present disclosure results in a reduced amount of immunosuppressive agents administered to the recipient when compared to the amount of immunosuppressive agents generally 57 administer to a comparable transplant recipient (e.g., a person of the same sex and of comparable age, height, and/or weight). In one embodiment, the method provided herein results in a reduced amount of immunosuppressive agents by 10% administered to the recipient. In one embodiment, the method provided herein results in a reduced amount of immunosuppressive agents by 10-20% administered to the recipient. In one embodiment, the method provided herein results in a reduced amount of immunosuppressive agents by 20-30% administered to the recipient. In one embodiment, the method provided herein results in a reduced amount of immunosuppressive agents by 30-40% administered to the recipient. In one embodiment, the method provided herein results in a reduced amount of immunosuppressive agents by 40-50% administered to the recipient. In one embodiment, the method provided herein results in a reduced amount of immunosuppressive agents by 50-60% administered to the recipient. In one embodiment, the method provided herein results in a reduced amount of immunosuppressive agents by 60-70% administered to the recipient. In one embodiment, the method provided herein results in a reduced amount of immunosuppressive agents by 70-80% administered to the recipient. In one embodiment, the method provided herein results in a reduced amount of immunosuppressive agents by 80-90% administered to the recipient. In one embodiment, the method provided herein results in a reduced amount of immunosuppressive agents by more than 90% administered to the recipient.

In some embodiments, xenotransplantation using a transplant produced according to the present disclosure results in reduced administration frequency of immunosuppressive therapy to the recipient when compared to the one which is typically administered to a comparable recipient (e.g., a person of the same sex and of comparable age, height, and/or weight). In one embodiment, the method provided herein results in a reduced frequency of immunosuppressive agents by 10% administered to the recipient. In one embodiment, the method provided herein results in a reduced frequency of immunosuppressive agents by 10-20% administered to the recipient. In one embodiment, the method provided herein results in a reduced frequency of immunosuppressive agents by 20-30% administered to the recipient. In one embodiment, the method provided herein results in a reduced frequency of immunosuppressive agents by 30-40% administered to the recipient. In one embodiment, the method provided herein results in a reduced frequency of immunosuppressive agents by 40-50% administered to the recipient. In one embodiment, the method provided herein results in a reduced frequency of immunosuppressive agents by 50-60% administered to the recipient. In one embodiment, the method provided herein results in a reduced frequency of immunosuppressive agents by 60-70% administered to the recipient. In one embodiment, the method provided herein results in a reduced frequency of immunosuppressive agents by 70-80% administered to the recipient. In one embodiment, the method provided herein results in a reduced frequency of immunosuppressive agents by 80-90% administered to the recipient. In one embodiment, the method provided herein results in a reduced frequency of immunosuppressive agents by more than 90% administered to the recipient.

In some embodiments, xenotransplantation using a transplant produced according to the present disclosure results in a shortened treatment duration of immunosuppressive therapy administered to the recipient when compared to the one which is typically administered to a comparable recipient (e.g., a person of the same sex and of comparable age, height, and/or weight). In one embodiment, the method provided herein results in a shortened treatment duration of immunosuppressive therapy by 10% administered to the recipient. In one embodiment, the method provided herein results in a shortened treatment duration of immunosuppressive therapy by 10-20% administered to the recipient. In one embodiment, the method provided herein results in a shortened treatment duration of immunosuppressive therapy by 20-30% administered to the recipient. In one embodiment, the method provided herein results in a shortened treatment duration of immunosuppressive therapy by 30-40% administered to the recipient. In one embodiment, the method provided herein results in a shortened treatment duration of immunosuppressive therapy by 40-50% administered to the recipient. In one embodiment, the method provided herein results in shortened treatment duration of immunosuppressive therapy by 50-60% administered to the recipient. In one embodiment, the method provided herein results in a shortened treatment duration of immunosuppressive therapy by 60-70% administered to the recipient. In one embodiment, the method provided herein results in a shortened treatment duration of immunosuppressive therapy by 70-80% administered to the recipient. In one embodiment, the method provided herein results in a shortened treatment duration of immunosuppressive therapy by 80-90% administered to the recipient. In one embodiment, the method provided herein results in a shortened treatment duration of immunosuppressive therapy by more than 90% administered to the recipient.

In some embodiments, xenotransplantation using a transplant produced according to the present disclosure results in decreased occurrences of rejection of the donor cells, tissues, and/or organs as described in Section 7.1 in a short term, when compared to the ones from the recombinant miniature swine where only CD47 is engineered (i.e., porcine CD47 gene is deleted or inactivated, and human CD47 gene is inserted). In other embodiments, a method of transplantation described herein results in decreased occurrences of rejection of the donor cells, tissues, and/or organs as described in Section 7.1 in a long term, when compared to the ones from the recombinant miniature swine where only CD47 is engineered (i.e., porcine CD47 gene is deleted or inactivated, and human CD47 gene is inserted).

In some embodiments, xenotransplantation using a transplant produced according to the present disclosure results in decreased occurrences of short term rejection of the donor cells, tissues, and/or organs (e.g., donor cells, tissues, and/or organs described in Section 7.1), when compared to allogeneic donor cells, tissues, and/or organs. In other embodiments, xenotransplantation using a transplant produced according to the present disclosure results in decreased occurrences of long-term rejection of the donor cells, tissues, and/or organs (e.g., donor cells, tissues, and/or organs described in Section 7.1), when compared to allogeneic donor cells, tissues, and/or organs.

In some embodiments, xenotransplantation using a transplant produced according to the present disclosure results in more prolonged viability of the donor cells, tissues, and/or organs obtained from the recombinant miniature swine described herein, when compared to donor cells, tissues, and/or organs obtained from recombinant miniature swine where only CD47 is engineered (i.e., porcine CD47 gene is deleted or inactivated, and human CD47 gene is inserted). In one embodiment, the method provided herein results in a viability of the donor cells, tissues, and/or organs prolonged by less than 10%. In one embodiment, the method provided herein results in a viability of the donor cells, tissues, and/or organs prolonged by less than 10-25%. In one embodiment, the method provided herein results in a viability of the donor cells, tissues, and/or organs prolonged by 25-50%. In one embodiment, the method provided herein results in a viability of the donor cells, tissues, and/or organs prolonged by 50-75%. In one embodiment, the method provided herein results in a viability of the donor cells, tissues, and/or organs prolonged by 75-100%. In one embodiment, the method provided herein results in a viability of the donor cells, tissues, and/or organs prolonged by 100-200%. In one embodiment, the method provided herein results in a viability of the donor cells, tissues, and/or organs prolonged by 200-300%. In one embodiment, the method provided herein results in a viability of the donor cells, tissues, and/or organs prolonged by more than 300%. In one embodiment, the method provided herein results in a viability of the donor cells, tissues, and/or organs prolonged by 1-5 more years. In one embodiment, the method provided herein results in a viability of the donor cells, tissues, and/or organs prolonged by 5-10 more years. In one embodiment, the method provided herein results in a viability of the donor cells, tissues, and/or organs prolonged by 10-15 more years. In one embodiment, the method provided herein results in a viability of the donor cells, tissues, and/or organs prolonged by 15-20 more years. In one embodiment, the method provided herein results in a viability of the donor cells, tissues, and/or organs prolonged by 20-25 more years. In one embodiment, the method provided herein results in a viability of the donor cells, tissues, and/or organs prolonged by 25-30 more years.

In other embodiments, xenotransplantation using a transplant produced according to the present disclosure results in more prolonged viability of the donor cells, tissues, and/or organs from such recombinant miniature swine as described in Section 7.1, when compared to allogeneic donor cells, tissues, and/or organs. In one embodiment, the method provided herein results in a viability of the donor cells, tissues, and/or organs prolonged by less than 10%. In one embodiment, the method provided herein results in a viability of the donor cells, tissues, and/or organs prolonged by less than 10-25%. In one embodiment, the method provided herein results in a viability of the donor cells, tissues, and/or organs prolonged by 25-50%. In one embodiment, the method provided herein results in a viability of the donor cells, tissues, and/or organs prolonged by 50-75%. In one embodiment, the method provided herein results in a viability of the donor cells, tissues, and/or organs prolonged by 75-100%. In one embodiment, the method provided herein results in a viability of the donor cells, tissues, and/or organs prolonged by 100-200%. In one embodiment, the method provided herein results in a viability of the donor cells, tissues, and/or organs prolonged by 200-300%. In one embodiment, the method provided herein results in a viability of the donor cells, tissues, and/or organs prolonged by more than 300%. In one embodiment, the method provided herein results in a viability of the donor cells, tissues, and/or organs prolonged by 1-5 more years. In one embodiment, the method provided herein results in a viability of the donor cells, tissues, and/or organs prolonged by 5-10 more years. In one embodiment, the method provided herein results in a viability of the donor cells, tissues, and/or organs prolonged by 10-15 more years. In one embodiment, the method provided herein results in a viability of the donor cells, tissues, and/or organs prolonged by 15-20 more years. In one embodiment, the method provided herein results in a viability of the donor cells, tissues, and/or organs prolonged by 20-25 more years. In one embodiment, the method provided herein results in a viability of the donor cells, tissues, and/or organs prolonged by 25-30 more years.

In some embodiments, xenotransplantation using a transplant produced according to the present disclosure enables the donor cells, tissues, and/or organs survive for in the recipient at least for 6 months. In other embodiments, the method provided herein enables the donor cells, tissues, and/or organs survive for in the recipient at least for 1 year. In other embodiments, the method provided herein enables the donor cells, tissues, and/or organs survive for in the recipient at least for 5 years. In other embodiments, the method provided herein enables the donor cells, tissues, and/or organs survive for in the recipient at least for 10 years. In other embodiments, the method provided herein enables the donor cells, tissues, and/or organs survive for in the recipient at least for 15 years. In other embodiments, the method provided herein enables the donor cells, tissues, and/or organs survive for in the recipient at least for 20 years.

In some embodiments, xenotransplantation using a transplant produced according to the present disclosure results in better function of the donor cells, tissues, and/or organs after transplantation, when compared to the ones from the recombinant miniature swine where only CD47 is engineered (i.e., porcine CD47 gene is deleted or inactivated, and human CD47 gene is inserted). In other embodiments, the method provided herein results in better function of the donor cells, tissues, and/or organs after transplantation, when compared to allogeneic donor cells, tissues, and/or organs.

In some embodiments, xenotransplantation using a transplant produced according to the present disclosure results in improvements of the corresponding disease that the transplantation aims to intervene with in the recipients who receive the donor cells, tissues, and/or organs, indicated by the corresponding biomarker(s), when compared to the ones from the recombinant miniature swine where only CD47 is engineered (i.e., porcine CD47 gene is deleted or inactivated, and human CD47 gene is inserted). In other embodiments, the method provided herein results in improvements of the corresponding disease that the transplantation aims to intervene with in the recipients who receive the donor cells, tissues, and/or organs, indicated by the corresponding biomarker(s), when compared to allogeneic donor cells, tissues, and/or organs.

In some embodiments, xenotransplantation using a transplant produced according to the present disclosure results in normal functions of other untargeted organ systems in the recipients who receive the donor cells, tissues, and/or organs from such recombinant miniature swine as described in Section 7.1, when compared to the recipients who receive the ones from the recombinant miniature swine where only CD47 is engineered (i.e., porcine CD47 gene is deleted or inactivated, and human CD47 gene is inserted). In other embodiments, the method provided herein results in normal functions of other untargeted organ systems in the recipients who receive the donor cells, tissues, and/or organs from such recombinant miniature swine as described in Section 7.1, when compared to the recipients who receive allogeneic donor cells, tissues, and/or organs.

In some embodiments, xenotransplantation using a transplant produced according to the present disclosure results in longer survival of the recipients who receive the donor cells, tissues, and/or organs from the recombinant miniature swine described herein (e.g., the recombinant miniature swine described in Section 7.1) when compared to a transplant from recombinant miniature swine where only CD47 is engineered (i.e., porcine CD47 gene is deleted or inactivated, and human CD47 gene is inserted). In one embodiment, the method provided herein results in longer survival of the recipients by less than 10%. In one embodiment, the method provided herein results in longer survival of the recipients by less than 10-25%. In one embodiment, the method provided herein results in longer survival of the recipients by 25-50%. In one embodiment, the method provided herein results in longer survival of the recipients by 50-75%. In one embodiment, the method provided herein results in longer survival of the recipients by 75-100%. In one embodiment, the method provided herein results in longer survival of the recipients by 100-200%. In one embodiment, the method provided herein results in longer survival of the recipients by 200-300%. In one embodiment, the method provided herein results in longer survival of the recipients by more than 300%. In one embodiment, the method provided herein results in a longer survival of the recipients by 1-5 more years. In one embodiment, the method provided herein results in longer survival of the recipients by 5-10 more years. In one embodiment, the method provided herein results in longer survival of the recipients by 10-15 more years. In one embodiment, the method provided herein results in longer survival of the recipients by 15-20 more years. In one embodiment, the method provided herein results in longer survival of the recipients by 20-25 more years. In one embodiment, the method provided herein results in longer survival of the recipients by 25-30 more years.

In some embodiments, xenotransplantation using a transplant produced according to the present disclosure results in longer survival of the recipients who receive the donor cells, tissues, and/or organs from the recombinant miniature swine described herein e.g., the recombinant miniature swine described in Section 7.1) when compared to allogeneic donor cells, tissues, and/or organs. In one embodiment, the method provided herein results in longer survival of the recipients by less than 10%. In one embodiment, the method provided herein results in longer survival of the recipients by less than 10-25%. In one embodiment, the method provided herein results in longer survival of the recipients by 25-50%. In one embodiment, the method provided herein results in longer survival of the recipients by 50-75%. In one embodiment, the method provided herein results in longer survival of the recipients by 75-100%. In one embodiment, the method provided herein results in longer survival of the recipients by 100-200%. In one embodiment, the method provided herein results in longer survival of the recipients by 200-300%. In one embodiment, the method provided herein results in longer survival of the recipients by more than 300%. In one embodiment, the method provided herein results in a longer survival of the recipients by 1-5 more years. In one embodiment, the method provided herein results in longer survival of the recipients by 5-10 more years. In one embodiment, the method provided herein results in longer survival of the recipients by 10-15 more years. In one embodiment, the method provided herein results in longer survival of the recipients by 15-20 more years. In one embodiment, the method provided herein results in longer survival of the recipients by 20-25 more years. In one embodiment, the method provided herein results in longer survival of the recipients by 25-30 more years.

In some embodiments, xenotransplantation using a transplant produced according to the present disclosure results in better quality of life of the recipients who receive the donor cells, tissues, and/or organs from such recombinant miniature swine as described in Section 7.1, when compared to the recipients who receive the ones from the recombinant miniature swine where only CD47 is engineered (i.e., porcine CD47 gene is deleted or inactivated, and human CD47 gene is inserted). In other embodiments, the method provided herein results in better quality of life of the recipients who receive the donor cells, tissues, and/or organs from such recombinant miniature swine as described in Section 7.1, when compared to the recipients who receive allogeneic donor cells, tissues, and/or organs.

In specific embodiments, xenotransplantation to baboons using donor cells, tissues, and/or organs from the recombinant miniature swine produced according to the present disclosure is more advantageous, relative to xenotransplantation to baboons using donor cells, tissues, and/or organs from a recombinant miniature swine where only CD47 is engineered (i.e., porcine CD47 gene is deleted or inactivated, and human CD47 gene is inserted). For example, in some embodiments, xenotransplantation to baboons using donor cells, tissues, and/or organs from the recombinant miniature swine produced according to the present disclosure results in reduced administration of immunosuppressive therapy, decreased occurrences of rejection of the donor cells, tissues, and/or organs in a short term, decreased occurrences of rejection of the donor cells, tissues, and/or organs in a long term, more prolonged viability, better function of the donor cells, tissues, and/or organs, normal functions of other untargeted organ systems, better survival of the baboons, better quality of life of the baboons, or a combination of any of the above, relative to xenotransplantation to baboons using donor cells, tissues, and/or organs from a recombinant miniature swine where only CD47 is engineered.

In another specific embodiments, xenotransplantation to humans using donor cells, tissues, and/or organs from the recombinant miniature swine produced according to the present disclosure is more advantageous, relative to transplantation using non-engineered donor cells, tissues, and/or organs. For example, in some embodiments xenotransplantation to humans using donor cells, tissues, and/or organs from the recombinant miniature swine produced according to the present disclosure results in reduced administration of immunosuppressive therapy, decreased occurrences of rejection of the donor cells, tissues, and/or organs in a short term, decreased occurrences of rejection of the donor cells, tissues, and/or organs in a long term, more prolonged viability, better function of the donor cells, tissues, and/or organs, improvements of the corresponding disease indicated by the corresponding biomarker(s), normal functions of other untargeted organ systems, better survival of the human recipients, better quality of life of the human recipients, or a combination of any of the above, relative to transplantation using non-engineered donor cells, tissues, and/or organs.

Proteinuria is characterized by increased levels of protein in the urine and can be a symptom of decreased kidney function and potentially renal failure. In yet other specific embodiments, if a kidney from such recombinant miniature swine as described in Section 7.1 is transplanted to a recipient, no proteinuria is observed. In yet other specific embodiments, if a kidney from such recombinant miniature swine as described in Section 7.1 is transplanted to a recipient, the severity of proteinuria is reduced.

In some embodiments, the severity of proteinuria is reduced by 10%, 20%, 30%, 40%, 50%, 60%, 70%, 80%, 90%, 95% or over 95%. In some embodiments, a patient treated in accordance with a method provided herein will not experience proteinuria, defined as the excretion or over 150 mg protein per day in the urine. In some embodiments, a patient treated in accordance with a method provided herein may experience transient proteinuria that resolves after 1, 2, 3, 3-7, 7-10, 10-14 days, or 1-2, 2-3, 3-4, 4-5, 5-6, 6-7, 7-8 weeks, or 1, 2, 3, 4, 5, 6 months after the transplantation.

In some embodiments, the concentration of total protein in the urine of a recipient treated with a method described herein developing proteinuria is less than about 60 mg per day, less than about 80 mg per day, less than about 100 mg per day, less than about 120 mg per day, less than about 140 mg per day, less than about 160 mg per day, less than about 200 mg per day, less than about 220 mg per day, less than about 240 mg, per day, less than about 260 mg per day, less than about 280 mg per day, less than about 300 mg per day, less than about 320 mg per day, less than about 340 mg per day, less than about 360 mg per day, less than about 380 mg per day or less than about 400 mg per day.

In some embodiments, the concentration of albumin in the urine of a recipient treated with a method described herein developing proteinuria is less than about 5 mg per day, less than about 10 mg per day, less than about 20 mg per day, less than about 30 mg per day, less than about 40 mg per day, less than about 50 mg per day, less than about 60 mg per day, less than about 70 mg per day, less than about 80 mg per day, less than about 90 mg per day or less than about 100 mg per day.

In some embodiments, the ratio of protein to creatinine in a 24 hour urine sample of a patient treated in accordance with the methods described herein is less than about 0.2, less than about 0.4, less than about 0.6, less than about 0.8 or less than about 1. In some embodiments, the ratio of albumin to creatinine in a 24 hour urine sample of a patient treated in accordance with the methods described herein is less than about 0.02, less than about 0.04, less than about 0.06, less than about 0.08 or less than about 0.1.

In some embodiments, the risk of a recipient treated with a method described herein developing proteinuria is decreased by about 10%, 20%, 30%, 40%, 50%, 60%, 70%, 80%, 90% or 95% compared to the risk of a recipient of a donor kidney in which only endogenous porcine CD47 is replaced with its human homolog.

TABLE 1
Sequences of Human CD47 and SIRPA

| SEQ | | |
|---|---|---|
| ID | | |
| NO. | Description | Sequence |
| 1 | Human CD47 coding | ATGTGGCCCCTGGTAGCGGCGCTGTTGCTGGGC |
| | sequence (CDS) of isoform | TCGGCGTGCTGCGGATCAGCTCAGCTACTATTT |
| | 1 | AATAAAACAAAATCTGTAGAATTCACGTTTTGT |
| | | AATGACACTGTCGTCATTCCATGCTTTGTTACTA |
| | | ATATGGAGGCACAAAACACTACTGAAGTATAC |
| | | GTAAAGTGGAAATTTAAAGGAAGAGATATTTA |
| | | CACCTTTGATGGAGCTCTAAACAAGTCCACTGT |
| | | CCCCACTGACTTTAGTAGTGCAAAAATTGAAGT |
| | | CTCACAATTACTAAAAGGAGATGCCTCTTTGAA |
| | | GATGGATAAGAGTGATGCTGTCTCACACACAG |
| | | GAAACTACACTTGTGAAGTAACAGAATTAACC |
| | | AGAGAAGGTGAAACGATCATCGAGCTAAAATA |
| | | TCGTGTTGTTTCATGGTTTTCTCCAAATGAAAAT |
| | | ATTCTTATTGTTATTTTCCCAATTTTTGCTATAC |
| | | TCCTGTTCTGGGGACAGTTTGGTATTAAAACAC |
| | | TTAAATATAGATCCGGTGGTATGGATGAGAAA |
| | | ACAATTGCTTTACTTGTTGCTGGACTAGTGATC |
| | | ACTGTCATTGTCATTGTTGGAGCCATTCTTTTCG |
| | | TCCCAGGTGAATATTCATTAAAGAATGCTACTG |
| | | GCCTTGGTTTAATTGTGACTTCTACAGGGATAT |
| | | TAATATTACTTCACTACTATGTGTTTAGTACAGC |
| | | GATTGGATTAACCTCCTTCGTCATTGCCATATTG |
| | | GTTATTCAGGTGATAGCCTATATCCTCGCTGTG |
| | | GTTGGACTGAGTCTCTGTATTGCGGCGTGTATA |
| | | CCAATGCATGGCCCTCTTCTGATTTCAGGTTTG |
| | | AGTATCTTAGCTCTAGCACAATTACTTGGACTA |
| | | GTTTATATGAAATTTGTGGCTTCCAATCAGAAG |
| | | ACTATACAACCTCCTAGGAAAGCTGTAGAGGA |
| | | ACCCCTTAATGCATTCAAAGAATCAAAAGGAAT |
| | | GATGAATGATGAATAA |
| 2 | Human CD47 CDS of | ATGTGGCCCCTGGTAGCGGCGCTGTTGCTGGGC |
| | isoform 2 | TCGGCGTGCTGCGGATCAGCTCAGCTACTATTT |
| | | AATAAAACAAAATCTGTAGAATTCACGTTTTGT |
| | | AATGACACTGTCGTCATTCCATGCTTTGTTACTA |
| | | ATATGGAGGCACAAAACACTACTGAAGTATAC |
| | | GTAAAGTGGAAATTTAAAGGAAGAGATATTTA |
| | | CACCTTTGATGGAGCTCTAAACAAGTCCACTGT |
| | | CCCCACTGACTTTAGTAGTGCAAAAATTGAAGT |
| | | CTCACAATTACTAAAAGGAGATGCCTCTTTGAA |
| | | GATGGATAAGAGTGATGCTGTCTCACACACAG |
| | | GAAACTACACTTGTGAAGTAACAGAATTAACC |
| | | AGAGAAGGTGAAACGATCATCGAGCTAAAATA |
| | | TCGTGTTGTTTCATGGTTTTCTCCAAATGAAAAT |
| | | ATTCTTATTGTTATTTTCCCAATTTTTGCTATAC |
| | | TCCTGTTCTGGGGACAGTTTGGTATTAAAACAC |
| | | TTAAATATAGATCCGGTGGTATGGATGAGAAA |
| | | ACAATTGCTTTACTTGTTGCTGGACTAGTGATC |
| | | ACTGTCATTGTCATTGTTGGAGCCATTCTTTTCG |
| | | TCCCAGGTGAATATTCATTAAAGAATGCTACTG |
| | | GCCTTGGTTTAATTGTGACTTCTACAGGGATAT |
| | | TAATATTACTTCACTACTATGTGTTTAGTACAGC |
| | | GATTGGATTAACCTCCTTCGTCATTGCCATATTG |
| | | GTTATTCAGGTGATAGCCTATATCCTCGCTGTG |
| | | GTTGGACTGAGTCTCTGTATTGCGGCGTGTATA |
| | | CCAATGCATGGCCCTCTTCTGATTTCAGGTTTG |
| | | AGTATCTTAGCTCTAGCACAATTACTTGGACTA |
| | | GTTTATATGAAATTTGTGGCTTCCAATCAGAAG |
| | | ACTATACAACCTCCTAGGAATAACTGA |
| 3 | Human CD47 CDS of | ATGTGGCCCCTGGTAGCGGCGCTGTTGCTGGGC |
| | isoform 3 | TCGGCGTGCTGCGGATCAGCTCAGCTACTATTT |
| | | AATAAAACAAAATCTGTAGAATTCACGTTTTGT |
| | | AATGACACTGTCGTCATTCCATGCTTTGTTACTA |
| | | ATATGGAGGCACAAAACACTACTGAAGTATAC |
| | | GTAAAGTGGAAATTTAAAGGAAGAGATATTTA |
| | | CACCTTTGATGGAGCTCTAAACAAGTCCACTGT |
| | | CCCCACTGACTTTAGTAGTGCAAAAATTGAAGT |
| | | CTCACAATTACTAAAAGGAGATGCCTCTTTGAA |
| | | GATGGATAAGAGTGATGCTGTCTCACACACAG |
| | | GAAACTACACTTGTGAAGTAACAGAATTAACC |
| | | AGAGAAGGTGAAACGATCATCGAGCTAAAATA |
| | | TCGTGTTGTTTCATGGTTTTCTCCAAATGAAAAT |
| | | ATTCTTATTGTTATTTTCCCAATTTTTGCTATAC |
| | | TCCTGTTCTGGGGACAGTTTGGTATTAAAACAC |
| | | TTAAATATAGATCCGGTGGTATGGATGAGAAA |
| | | ACAATTGCTTTACTTGTTGCTGGACTAGTGATC |
| | | ACTGTCATTGTCATTGTTGGAGCCATTCTTTTCG |
| | | TCCCAGGTGAATATTCATTAAAGAATGCTACTG |
| | | GCCTTGGTTTAATTGTGACTTCTACAGGGATAT |
| | | TAATATTACTTCACTACTATGTGTTTAGTACAGC |
| | | GATTGGATTAACCTCCTTCGTCATTGCCATATTG |
| | | GTTATTCAGGTGATAGCCTATATCCTCGCTGTG |
| | | GTTGGACTGAGTCTCTGTATTGCGGCGTGTATA |
| | | CCAATGCATGGCCCTCTTCTGATTTCAGGTTTG |
| | | AGTATCTTAGCTCTAGCACAATTACTTGGACTA |
| | | GTTTATATGAAATTTGTGGCTTCCAATCAGAAG |
| | | ACTATACAACCTCCTAGGAAAGCTGTAGAGGA |
| | | ACCCCTTAATGAATAA |
| 4 | Human SIRPA CDS of | ATGGAGCCCGCCGGCCCGGCCCCCGGCCGCCTC |
| | isoform 1 | GGGCCGCTGCTCTGCCTGCTGCTCGCCGCGTCC |
| | | TGCGCCTGGTCAGGAGTGGCGGGTGAGGAGGA |
| | | GCTGCAGGTGATTCAGCCTGACAAGTCCGTGTT |
| | | GGTTGCAGCTGGAGAGACAGCCACTCTGCGCTG |
| | | CACTGCGACCTCTCTGATCCCTGTGGGGCCCAT |
| | | CCAGTGGTTCAGAGGAGCTGGACCAGGCCGGG |
| | | AATTAATCTACAATCAAAAAGAAGGCCACTTCC |
| | | CCCGGGTAACAACTGTTTCAGACCTCACAAAGA |
| | | GAAACAACATGGACTTTTCCATCCGCATCGGTA |
| | | ACATCACCCCAGCAGATGCCGGCACCTACTACT |
| | | GTGTGAAGTTCCGGAAAGGGAGCCCCGATGAC |
| | | GTGGAGTTTAAGTCTGGAGCAGGCACTGAGCTG |
| | | TCTGTGCGCGCCAAACCCTCTGCCCCCGTGGTA |
| | | TCGGGCCCTGCGGCGAGGGCCACACCTCAGCA |
| | | CACAGTGAGCTTCACCTGCGAGTCCCACGGCTT |
| | | CTCACCCAGAGACATCACCCTGAAATGGTTCAA |
| | | AAATGGGAATGAGCTCTCAGACTTCCAGACCA |
| | | ACGTGGACCCCGTAGGAGAGAGCGTGTCCTAC |
| | | AGCATCCACAGCACAGCCAAGGTGGTGCTGAC |
| | | CCGCGAGGACGTTCACTCTCAAGTCATCTGCGA |
| | | GGTGGCCCACGTCACCTTGCAGGGGGACCCTCT |
| | | TCGTGGGACTGCCAACTTGTCTGAGACCATCCG |
| | | AGTTCCACCCACCTTGGAGGTTACTCAACAGCC |
| | | CGTGAGGGCAGAGAACCAGGTGAATGTCACCT |
| | | GCCAGGTGAGGAAGTTCTACCCCCAGAGACTA |
| | | CAGCTGACCTGGTTGGAGAATGGAAACGTGTCC |
| | | CGGACAGAAACGGCCTCAACCGTTACAGAGAA |
| | | CAAGGATGGTACCTACAACTGGATGAGCTGGCT |
| | | CCTGGTGAATGTATCTGCCCACAGGGATGATGT |
| | | GAAGCTCACCTGCCAGGTGGAGCATGACGGGC |
| | | AGCCAGCGGTCAGCAAAAGCCATGACCTGAAG |
| | | GTCTCAGCCCACCCGAAGGAGCAGGGCTCAAA |
| | | TACCGCCGCTGAGAACACTGGATCTAATGAACG |
| | | GAACATCTATATTGTGGTGGGTGTGGTGTGCAC |
| | | CTTGCTGGTGGCCCTACTGATGGCGGCCCTCTA |
| | | CCTCGTCCGAATCAGACAGAAGAAAGCCCAGG |
| | | GCTCCACTTCTTCTACAAGGTTGCATGAGCCCG |
| | | AGAAGAATGCCAGAGAAATAACACAGGACACA |
| | | AATGATATCACATATGCAGACCTGAACCTGCCC |
| | | AAGGGGAAGAAGCCTGCTCCCCAGGCTGCGGA |
| | | GCCCAACAACCACACGGAGTATGCCAGCATTC |
| | | AGACCAGCCCGCAGCCCGCGTCGGAGGACACC |
| | | CTCACCTATGCTGACCTGGACATGGTCCACCTC |
| | | AACCGGACCCCCAAGCAGCCGGCCCCCAAGCC |
| | | TGAGCCGTCCTTCTCAGAGTACGCCAGCGTCCA |
| | | GGTCCCGAGGAAGTGA |
| 5 | Human SIRPA CDS of | ATGGAGCCCGCCGGCCCGGCCCCCGGCCGCCTC |
| | isoform 2 | GGGCCGCTGCTCTGCCTGCTGCTCGCCGCGTCC |
| | | TGCGCCTGGTCAGGAGTGGCGGGTGAGGAGGA |
| | | GCTGCAGGTGATTCAGCCTGACAAGTCCGTGTT |
| | | GGTTGCAGCTGGAGAGACAGCCACTCTGCGCTG |
| | | CACTGCGACCTCTCTGATCCCTGTGGGGCCCAT |
| | | CCAGTGGTTCAGAGGAGCTGGACCAGGCCGGG |
| | | AATTAATCTACAATCAAAAAGAAGGCCACTTCC |
| | | CCCGGGTAACAACTGTTTCAGACCTCACAAAGA |
| | | GAAACAACATGGACTTTTCCATCCGCATCGGTA |
| | | ACATCACCCCAGCAGATGCCGGCACCTACTACT |
| | | GTGTGAAGTTCCGGAAAGGGAGCCCCGATGAC |
| | | GTGGAGTTTAAGTCTGGAGCAGGCACTGAGCTG |
| | | TCTGTGCGCGCCAAACCCTCTGCCCCCGTGGTA |
| | | TCGGGCCCTGCGGCGAGGGCCACACCTCAGCA |
| | | CACAGTGAGCTTCACCTGCGAGTCCCACGGCTT |
| | | CTCACCCAGAGACATCACCCTGAAATGGTTCAA |
| | | AAATGGGAATGAGCTCTCAGACTTCCAGACCA |
| | | ACGTGGACCCCGTAGGAGAGAGCGTGTCCTAC |
| | | AGCATCCACAGCACAGCCAAGGTGGTGCTGAC |
| | | CCGCGAGGACGTTCACTCTCAAGTCATCTGCGA |
| | | GGTGGCCCACGTCACCTTGCAGGGGGACCCTCT |
| | | TCGTGGGACTGCCAACTTGTCTGAGACCATCCG |
| | | AGTTCCACCCACCTTGGAGGTTACTCAACAGCC |
| | | CGTGAGGGCAGAGAACCAGGTGAATGTCACCT |
| | | GCCAGGTGAGGAAGTTCTACCCCCAGAGACTA |
| | | CAGCTGACCTGGTTGGAGAATGGAAACGTGTCC |
| | | CGGACAGAAACGGCCTCAACCGTTACAGAGAA |
| | | CAAGGATGGTACCTACAACTGGATGAGCTGGCT |
| | | CCTGGTGAATGTATCTGCCCACAGGGATGATGT |
| | | GAAGCTCACCTGCCAGGTGGAGCATGACGGGC |
| | | AGCCAGCGGTCAGCAAAAGCCATGACCTGAAG |
| | | GTCTCAGCCCACCCGAAGGAGCAGGGCTCAAA |
| | | TACCGCCGCTGAGAACACTGGATCTAATGAACG |
| | | GAACATCTATATTGTGGTGGGTGTGGTGTGCAC |
| | | CTTGCTGGTGGCCCTACTGATGGCGGCCCTCTA |
| | | CCTCGTCCGAATCAGACAGAAGAAAGCCCAGG |
| | | GCTCCACTTCTTCTACAAGGTTGCATGAGCCCG |
| | | AGAAGAATGCCAGAGAAATAACACAGGTACAG |
| | | TCCTTGGACACAAATGATATCACATATGCAGAC |
| | | CTGAACCTGCCCAAGGGGAAGAAGCCTGCTCC |
| | | CCAGGCTGCGGAGCCCAACAACCACACGGAGT |
| | | ATGCCAGCATTCAGACCAGCCCGCAGCCCGCGT |
| | | CGGAGGACACCCTCACCTATGCTGACCTGGACA |
| | | TGGTCCACCTCAACCGGACCCCCAAGCAGCCGG |
| | | CCCCCAAGCCTGAGCCGTCCTTCTCAGAGTACG |
| | | CCAGCGTCCAGGTCCCGAGGAAGTGA |

### 8. EXAMPLES

The following is a description of illustrative protocols that can be used to demonstrate the benefits of the methods and compositions described herein. These protocols are not intended to limit the scope of what the inventors regard as their disclosure nor are they intended to represent that the experiments below were performed and are all of the experiments that may be performed.

### 8.1 Example 1

Recombinant miniature swine are generated by replacing endogenous porcine CD47 and SIRPA with human homologs. Briefly, fetus fibroblast cells are collected from α-1,3-galactosyltransferase knockout miniature swine. The cells are transfected with (i) a vector carrying human CD47 CDS sequence, flanked by two homologous DNA arms of porcine CD47 sequences required for homologous recombination; (ii) a vector carrying human SIRPA CDS sequence, flanked by two homologous DNA arms of porcine SIRPA sequences required for homologous recombination; (iii) a vector carrying Cas9; (iv) a vector carrying an sgRNA targeting the mouse CD47 gene; and (v) a vector carrying an sgRNA targeting the mouse SIRPA gene. Before the transfection, the above vectors are confirmed by sequencing analysis. After transfection, the cells are cultured in media for recovery. After the recovery, the cells are split into single cells and cultured for more days to form colonies. Positive colonies are selected by PCR and sequencing analysis to identify the successful replacement of the endogenous porcine CD47 and SIRPA with human homologs. Miniature swine oocytes are collected for somatic cell nuclear transfers. Denuded oocytes are enucleated, and the cells from the above-mentioned positive colonies are used as donor cells for injection into the perivitelline space of oocytes. DC pulses are used for cell fusion. Reconstructed embryos are surgically transferred into the oviduct of surrogate swine, which deliver recombinant miniature swine for xenotransplantation. Sequencing analysis is conducted to confirm that the endogenous porcine CD47 and SIRPA are indeed substituted by their human homologs in the resulting recombinant miniature swine.

Kidneys from the above-mentioned recombinant miniature swine, where endogenous porcine CD47 and SIRPA are replaced with human homologs, are collected and transplanted into recipient baboons, along with bone marrow from the same miniature swine. In comparison, kidneys from recombinant miniature swine, where only endogenous porcine CD47 is replaced with its human homolog, are collected and transplanted into another group of recipient baboons, along with bone marrow from the same miniature swine. The following parameters are measured and compared between the two groups: (i) proteinuria, assessed by measuring urinary protein concentration after transplanting; (ii) survival duration of the transplanted kidneys without evidence of rejection; (iii) inflammatory reactions, such as systemic edema and cytokine levels (e.g., IL-6) in serum, together with comprehensive physical examinations of other organ systems in the recipient baboons; (iv) longevity of the recipient baboons; and (v) post-mortem analysis of the transplanted kidney for any possible atrophy and abnormalities.

### 8.2 Example 2—Humanized Porcine CD47

The following example demonstrates that porcine CD47 can be humanized by replacement of a portion of porcine CD47 with a portion of human CD47. Specifically, porcine CD47 was humanized by replacement of porcine exon 2 with human exon 2 to allow expression of the humanized CD47 from the native pig CD47 promoter under proper lineage and temporal expression patterns in transgenic pigs.

Briefly, the homologous recombination vector 47X2R for effecting exon 2 replacement was developed. The 47X2R vector was comprised of the last 1000 bp of pig CD47 intron 1, followed by exon 2 of human CD47, followed by the first 1020 bp of pig intron 2 (SEQ ID NO:6). Silent nucleotide substitutions of human CD47 exon 2 (G to A at position 1095 of the vector and CAGA to ACGC beginning at position 1314) were made to prevent CRISPR/Cas9 cutting at potential CRISPR guide sites within the vector.

Three potential RNA guide pairs near the junction of intron 1 and exon 2 were identified for use as RNP complexes in conjunction with Cas9 D10A nickase for introducing double-stranded breaks into the pig CD47 gene. Guide pair sequences tested, referenced to SEQ ID NO:7, were: (1) 47US-F1 (nt 43-63) and 47US-R1 (complement of nt 6-25); (2) 47US-F3 (nt 138-157) and 47US-R3 (complement of nt 95-114); and (3) 47US-F3 and 47US-R4 (complement of nt 113-132).

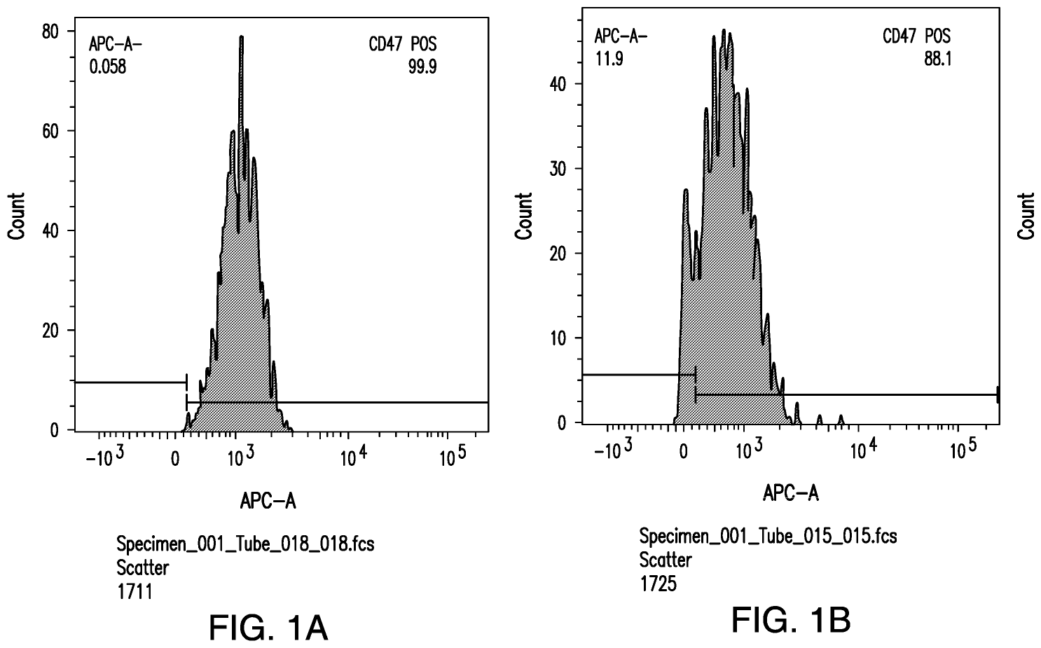

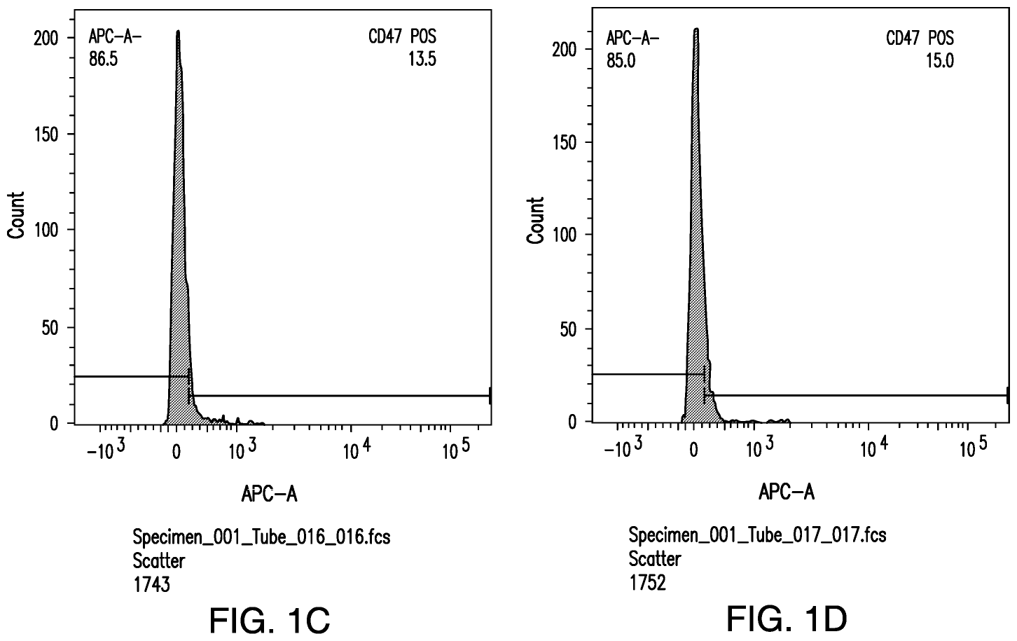

RNP complexes comprised of the above guide pairs and Cas9 D10A were nucleofected into porcine fetal fibroblasts and assessed for efficiency of loss of CD47 expression resulting from simultaneous loss of function at both CD47 alleles using FACS analysis of transfected cells stained with anti-CD47 monoclonal antibody CC2C6. Guide pairs 47US-F3/47US-R3 and 47US-F3/47US-R4 both resulted in high percentages of CD47 null cells (FIG. 1C and FIG. 1D, Table 2).

TABLE 2

| | CD47 Positive (%) |
|---|---|
| No Guides | 99.9 |
| Pair #1 (47US-F1 and 47US-R1) | 88.1 |
| Pair #2 (47US-F3 and 47US-R3) | 13.5 |
| Pair #3 (47US-F3 and 47US-R4) | 15.0 |

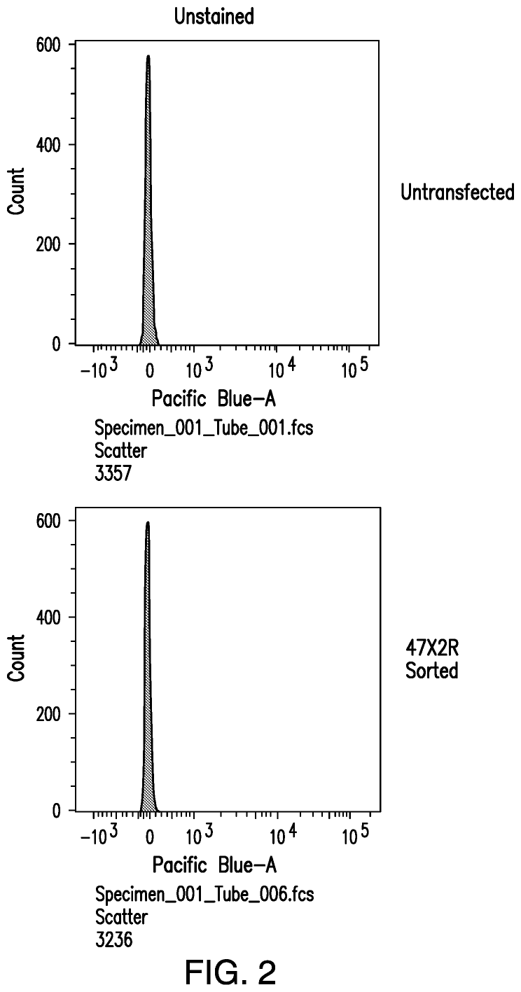

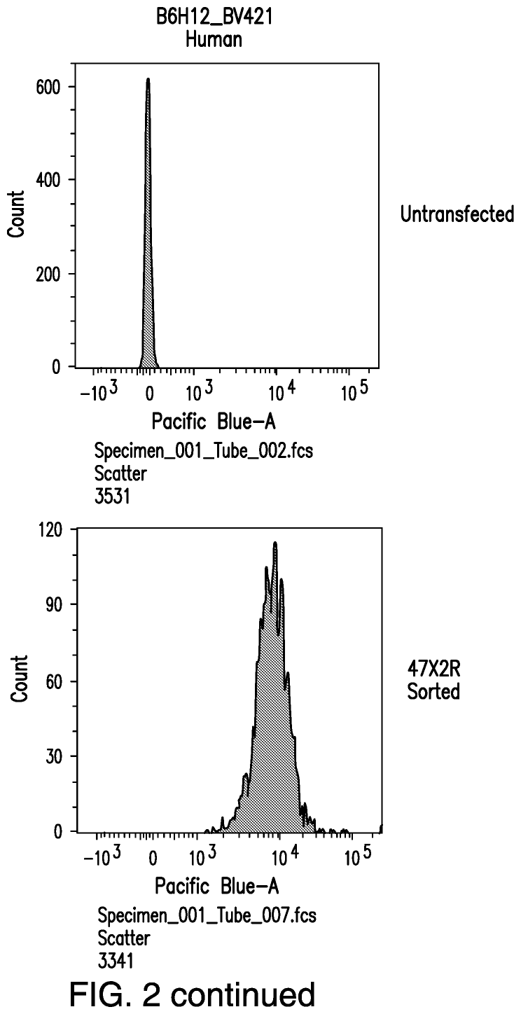

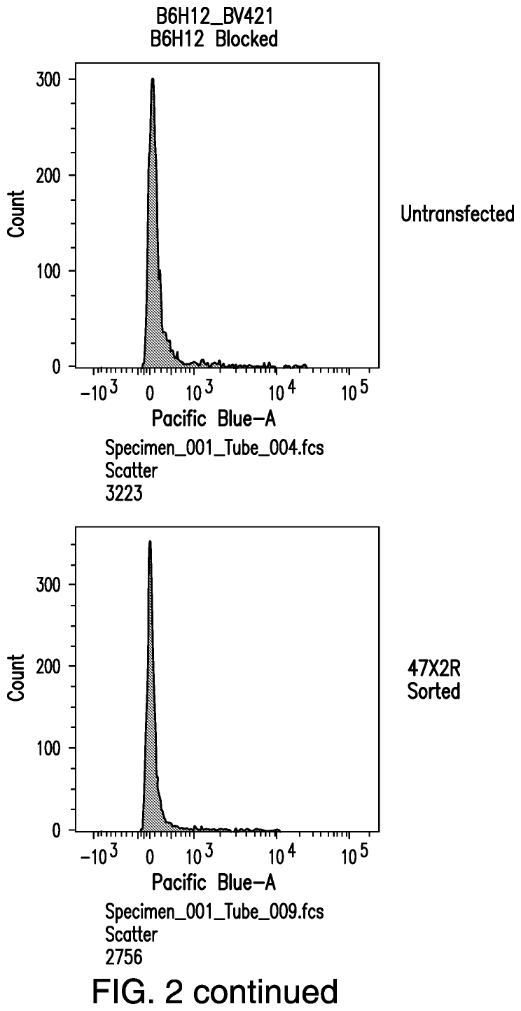

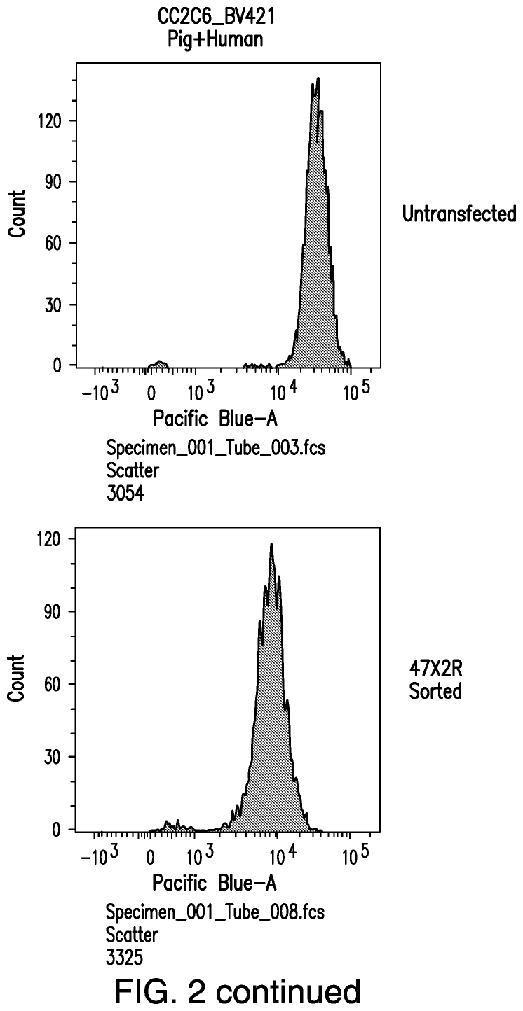

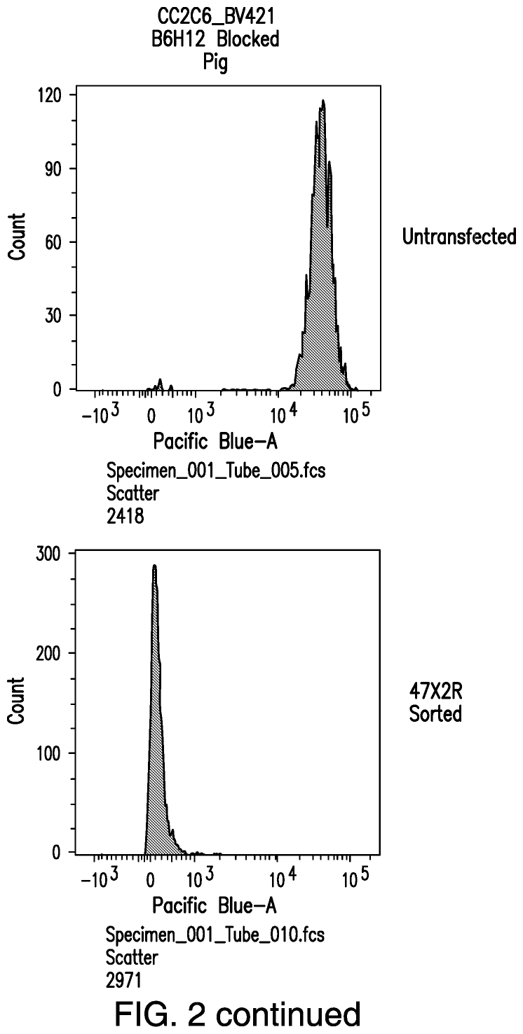

Next, homologous replacement of CD47 Exon 2 was performed using vector 47X2R and CRISPR/Cas9 D10A. The guide pair 47US-F3/47US-R3 was used as an exemplary set of guide pairs. Specifically, vector and RNP complexes were nucleofected into fibroblasts from a miniature swine fetus transgenic for human CD55 and CD59. Transfected cells were sorted based on binding to anti-CD47 monoclonal antibody B6H12, which binds human, but not porcine, CD47. Analysis of the sorted population demonstrated that a very high percentage of the population expressed humanized CD47. Staining with anti-CD47 monoclonal antibody CC26, which binds both human and pig CD47, when blocked with human specific monoclonal B6H12 further demonstrated that a very high percentage of the population was null for porcine CD47 (most probably due to mutations introduced by Cas9 cleavage of the non-targeted CD47 allele (FIG. 2). Loss of porcine CD47 function is a desired outcome of this modification.

Taken together, this example demonstrates that porcine CD47 can be humanized by introducing a portion of human CD47 (e.g., human exon 2 of CD47) into a homologous region in porcine CD47.

### 8.3 Example 3—Humanized Porcine SIRPA

Humanized porcine CD47 cells will be used as donor cells in somatic cell nuclear transfer to generate humanized CD47 fetuses. In this process, the nuclei of somatic cells are transferred into enucleated oocytes (e.g., metaphase II oocytes), and then this complex is activated. Reconstructed embryos are then cultured and transferred to synchronized recipients for gestation. This population is then subjected to genomic, RNA (e.g., RT-PCR), and/or protein analyses to confirm, for example, the expected structure, RNA, and protein expression of the transgenic locus and to determine if the porcine CD47 has been altered in the process.

The CD47 fetuses will be the starting material for humanization of the SIRPA gene using similar methods as described in Example 2.

### 9. EQUIVALENTS

Although the disclosure is described in detail with reference to specific embodiments thereof, it will be understood that variations that are functionally equivalent are within the scope of this disclosure. Indeed, various modifications of the disclosure in addition to those shown and described herein will become apparent to those skilled in the art from the foregoing description. Such modifications are intended to fall within the scope of the appended claims. Those skilled in the art will recognize, or be able to ascertain using no more than routine experimentation, many equivalents to the specific embodiments of the disclosure described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A recombinant miniature swine, wherein the recombinant miniature swine comprises:
(a) (i) a deleted or functionally inactivated endogenous gene encoding porcine CD47, wherein expression of the endogenous gene encoding porcine CD47 is regulated by porcine CD47 regulatory elements; and
(ii) a transgene encoding human CD47 inserted within porcine genome, wherein expression of the transgene encoding human CD47 is regulated by the porcine CD47 regulatory elements; and
(b) (i) a deleted or functionally inactivated endogenous gene encoding porcine SIRPα, wherein expression of the endogenous gene encoding porcine SIRPα is regulated by porcine SIRPA regulatory elements; and
(ii) a transgene encoding human SIRPα inserted within porcine genome, wherein expression of the transgene encoding human SIRPα is regulated by the porcine SIRPA regulatory elements.

2. The recombinant miniature swine of claim 1, wherein the miniature swine is an alpha-1,3 galactosyltransferase-deficient miniature swine.

3. The recombinant miniature swine of claim 2, wherein the alpha-1,3 galactosyltransferase-deficient miniature swine is a major histocompatibility complex (MHC)-inbred miniature swine.

4. The recombinant miniature swine of claim 1, wherein the human CD47 comprises SEQ ID NO. 1, 2, or 3.

5. The recombinant miniature swine of claim 1, wherein the human SIRPA comprises SEQ ID NO. 4 or 5.

6. The recombinant miniature swine of claim 1, wherein the transgenes encoding human SIRPα and human CD47 are inserted in the genome of the miniature swine by homologous recombination.

7. The recombinant miniature swine of claim 1, wherein the transgenes encoding human SIRPα and human CD47 are inserted in the porcine genome by non-homology-directed end-joining.

8. The recombinant miniature swine of claim 1, wherein the transgenes encoding human SIRPα and human CD47 are inserted in the porcine genome by recombinase-mediated cassette exchange.

9. The recombinant miniature swine of any one of claims 6 to 8, wherein the transgenes encoding human SIRPα and human CD47 are inserted in the porcine genome by a site-specific nuclease.

10. The recombinant miniature swine of claim 9, wherein the site-specific nuclease is selected from the group consisting of zinc fingers, a ZFN dimer, a ZFNickase, transcription activator-like effector nucleases (TALENs), and CRISPR/Cas9.

11. The recombinant miniature swine of claim 1, wherein expression of the transgene encoding human CD47 is regulated by the porcine CD47 regulatory elements of NCBI Gene ID: 397042.

12. The recombinant miniature swine of claim 1, wherein expression of the transgene encoding human SIRPα is regulated by the porcine SIRPA regulatory elements of NCBI Gene ID: 494566.

13. The recombinant miniature swine of any one of claims 1 to 3, wherein human CD47 protein expression is substantially similar to an expression pattern of endogenous porcine CD47, as determined by immunohistochemistry.

14. The recombinant miniature swine of any one of claims 1 to 3, wherein human SIRPα protein expression is substantially similar to an expression pattern of endogenous porcine SIRPα, as determined by immunohistochemistry.

15. A recombinant miniature swine, wherein the recombinant miniature swine comprises:
(a) a humanized CD47 gene; and/or
(b) a humanized SIRPA gene.

16. The recombinant miniature swine of claim 15, wherein the humanized CD47 gene comprises a porcine CD47 gene comprising exon 2 of human CD47.

17. A cell derived from the recombinant miniature swine in any one of claims 1 to 16.

18. An oocyte derived from the recombinant miniature swine in any one of claims 1 to 16.

19. A sperm derived from the recombinant miniature swine in any one of claims 1 to 16.

20. A tissue derived from the recombinant miniature swine in any one of claims 1 to 16.

21. An organ derived from the recombinant miniature swine in any one of claims 1 to 16.

22. A method of transplanting a graft from a first recombinant miniature swine to a primate, wherein the method comprises:
(a) obtaining the graft from the first recombinant miniature swine of any one of claims 1 to 16; and
(b) transplanting the graft to the primate.

23. The method of claim 22, wherein the primate is a human.

24. The method of claim 22, wherein graft comprises cells, a tissue, or an organ.

25. The method of claim 24, wherein the organ is selected from the group consisting of a heart, a kidney, an islet, a liver, a pancreas, a lung, an intestine, skin, a trachea, and a cornea, or a combination thereof.

26. The method of any one of claims 22 to 25, wherein the method further comprises obtaining bone marrow from a second recombinant miniature swine of any one of claims 1 to 16, and transplanting the bone marrow to the same primate.

27. The method of claim 26, wherein the bone marrow is transplanted at least 28 days before the graft from the first recombinant swine.

28. The method of claim 26, wherein the first recombinant miniature swine and the second recombinant miniature swine are the same recombinant swine.

29. The method of claim 26, wherein the first recombinant miniature swine and the second recombinant miniature swine are from highly inbred herd of miniature swine.

30. The method of claim 26, wherein the first recombinant miniature swine and the second recombinant miniature swine are genetically matched miniature swine.

31. The method of claim 26, wherein the first recombinant miniature swine and the second recombinant miniature swine are MHC matched.

32. The method of any one of claims 22 to 31, wherein the graft from the first recombinant miniature swine survives in the recipient at least for 6 months, 1 year, 5 years, 10 years, 15 years, or 20 years.

33. The method of any one of claims 22 to 31, wherein the graft from the first recombinant miniature swine functions in the recipient at least for 6 months, 1 year, 5 years, 10 years, 15 years, or 20 years.

34. The method of any one of claims 22 to 31, wherein immunosuppressive therapies that are needed in the recipient are reduced by 90%, 80%, 70%, 60%, or 50%.
